(19) United States
(12) Patent Application Publication
(10) Pub. No.: US 2009/0105119 A1
(43) Pub. Date: Apr. 23, 2009

(54) ASPARAGINE-10-SUBSTITUTED NONADEPSIPEPTIDES

(76) Inventors: Franz VON NUSSBAUM, Duesseldorf (DE); Nina Brunner, Essen (DE); Rainer Endermann, Wuppertal (DE); Joachim Telser, Wuppertal (DE); Joachim Schuhmacher, Wuppertal (DE); Sonja Anlauf, Wuppertal (DE)

Correspondence Address: MORRISON & FOERSTER LLP, 12531 HIGH BLUFF DRIVE, SUITE 100, SAN DIEGO, CA, 92130-2040, US

(21) Appl. No.: 12/180,507

(22) Filed: Jul. 25, 2008

Related U.S. Application Data
(63) Continuation of application No. PCT/EP2007/000645, filed on Jan. 25, 2007.

(30) Foreign Application Priority Data
Jan. 25, 2006 (DE) 10 2006 003 443.0

(51) Int. Cl. A61K 38/02; C07K 2/00; C07K 1/00

(52) U.S. Cl. 514/2; 530/300; 530/333

(57) ABSTRACT

The invention relates to nonadepsipeptides and methods for their preparation, as well as to their use for manufacturing medicaments for the treatment and/or prophylaxis of diseases, in particular bacterial infectious diseases.

### CROSS-REFERENCE TO RELATED APPLICATIONS

This application is a continuation of pending international application PCT/EP2007/000645, filed Jan. 25, 2007, designating US, which claims priority from German patent application DE 10 2006 003 443.0, filed Jan. 25, 2006. The contents of these documents are incorporated herein by reference in their entirety.

### BACKGROUND OF THE INVENTION

The invention relates to nonadepsipeptides and methods for their preparation, as well as to their use for manufacturing medicaments for the treatment and/or prophylaxis of diseases, in particular bacterial infectious diseases.

The bacterial cell wall is synthesized by a number of enzymes (cell wall biosynthesis) and is essential for the survival and reproduction of microorganisms. The structure of this macromolecule, as well as the proteins involved in the synthesis thereof, are highly conserved within the bacteria. Owing to its essential nature and uniformity, cell wall biosynthesis is an ideal point of attack for novel antibiotics (D. W. Green, The bacterial cell wall as a source of antibacterial targets, Expert Opin. Ther. Targets, 2002, 6, 1-19).

Vancomycin and penicillins are inhibitors of the bacterial cell wall biosynthesis and represent successful examples of the antibiotic potency of this principle of action. They have been employed for several decades clinically for the treatment of bacterial infections, especially with Gram-positive pathogens. Due to the growing occurrence of resistant microbes, e.g. methicillin-resistant staphylococci, penicillin-resistant pneumococci and vancomycin-resistant enterococci (F. Baquero, Gram-positive resistance: challenge for the development of new antibiotics, J. Antimicrob. Chemother., 1997, 39, Suppl A:1-6; A. P. Johnson, D. M. Livermore, G. S. Tillotson, Antimicrobial susceptibility of Gram-positive bacteria: what's current, what's anticipated?, J. Hosp. Infect., 2001, (49), Suppl A: 3-11) as well as recently also for the first time vancomycin-resistant staphylococci (B. Goldrick, First reported case of VRSA in the United States, Am. J. Nurs., 2002, 102, 17) these substances are increasingly losing their therapeutic efficacy.

The present invention describes a novel class of cell wall biosynthesis inhibitors without cross resistances with known antibiotic classes, as well as methods for their preparation.

Complex protecting-group operations are often a precondition for the semisynthetic derivatization of complex natural products (Haebich et al., Angew. Chem. Int. Ed., 2006, 45, 5072). Regio- and chemoselective addressing of the derivatization position is only possible in this way. In the present invention, surprisingly, a method which permits highly regio- and chemoselective derivatization of the complex depsipeptide lysobactin without protecting-group operations has been found.

The natural product lysobactin and some derivatives are described as having antibacterial activity in U.S. Pat. No. 4,754,018. The isolation and antibacterial activity of lysobactin is also described in EP-A-196 042 and JP 01132600. WO 04/099239 describes derivatives of lysobacin having antibacterial activity.

The antibacterial activity of lysobactin and katanosin A is furthermore described in O'Sullivan, J. et al., J. Antibiot. 1988, 41, 1740-1744, Bonner, D. P. et al., J. Antibiot. 1988, 41, 1745-1751, Shoji, J. et al., J. Antibiot. 1988, 41, 713-718 and Tymiak, A. A. et al., J. Org. Chem. 1989, 54, 1149-1157.

### SUMMARY OF THE INVENTION

One object of the present invention is to provide alternative compounds with comparable or improved antibacterial activity, better solubility, higher free fraction in blood plasma and better tolerability, e.g. less nephrotoxicity, for the treatment of bacterial diseases in humans and animals.

The invention relates to compounds of formula

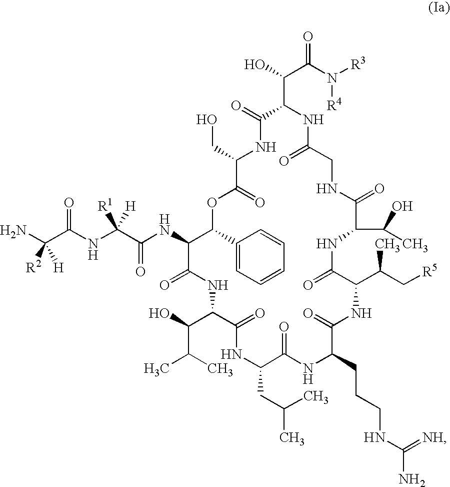

in which
R¹ represents 2-methylprop-1-yl, 2,2-dimethylprop-1-yl, 1-trimethylsilylmethyl or 3-pyridylmethyl,
   whereby 3-pyridylmethyl may be substituted with a trifluoromethyl substituent,
R² represents 2-methylprop-1-yl, 2,2-dimethylprop-1-yl, 2,2-dimethylbut-1-yl, 2-ethyl-2-methylbut-1-yl, 2,2-diethylbut-1-yl, 2,2-dimethylpent-1-yl or 1-trimethylsilylmethyl,
R³ represents C₁-C₆-alkyl,
   whereby alkyl may be substituted with 1 to 3 substituents, whereby the substituents are selected independently of one another from the group consisting of hydroxy, amino, hydroxycarbonyl, aminocarbonyl, {[bis(dimethylamino)methylene]amino}ethoxy, phenyl, 5- or 6-membered heterocyclyl, 5- or 6-membered heteroaryl, benzyloxycarbonyl and benzyloxycarbonylamino,
R⁴ represents hydrogen, C₁-C₄-alkyl, cyclopropyl or cyclopropylmethyl,
R⁵ represents hydrogen or methyl,
   and the salts thereof, the solvates thereof and the solvates of the salts thereof.

Compounds of the invention are compounds of formulae (Ia), (I), (IIa) and (II) and the salts, solvates, solvates of the salts and prodrugs thereof, the compounds which are emcompassed by formulae (Ia), (I), (IIa) and (II) and are of the formulae mentioned below, and the salts, solvates, solvates of the salts and prodrugs thereof, as well as the compounds which are encompassed by formulae (Ia), (I), (IIa) and (II) and are mentioned below as exemplary embodiments, and the salts, solvates, solvates of the salts and prodrugs thereof, insofar as the compounds which are encompassed by formulae (Ia), (I), (IIa) and (II) and are mentioned below are not already salts, solvates, solvates of the salts and prodrugs.

The compounds of the invention may, depending on their structure, exist in stereoisomeric forms (enantiomers, diastereomers). The invention therefore relates to the enantiomers or diastereomers and respective mixtures thereof. The stereoisomerically pure constituents can be isolated from such mixtures of enantiomers and/or diastereomers in a known manner.

Where the compounds of the invention can exist in tautomeric forms, the present invention encompasses all tautomeric forms.

Salts preferred for the purposes of the present invention are physiologically acceptable salts of the compounds of the invention. However, also included are salts which are not themselves suitable for pharmaceutical applications but can be used for example for the isolation or purification of the compounds of the invention. The term “salts” also encompasses mixed salts of the compounds according to the invention, such as mesylate trifluoroacetate salts for example.

Physiologically acceptable salts of the compounds of the invention include acid addition salts of mineral acids, carboxylic acids and sulfonic acids, e.g. salts of hydrochloric acid, hydrobromic acid, sulfuric acid, phosphoric acid, methanesulfonic acid, ethanesulfonic acid, toluenesulfonic acid, benzenesulfonic acid, naphthalenedisulfonic acid, acetic acid, trifluoroacetic acid, propionic acid, lactic acid, tartaric acid, malic acid, citric acid, fumaric acid, maleic acid and benzoic acid.

Physiologically acceptable salts of the compounds of the invention also include salts of usual bases such as, by way of example and preferably, alkali metal salts (e.g. sodium and potassium salts), alkaline earth metal salts (e.g. calcium and magnesium salts) and ammonium salts derived from ammonia or organic amines having 1 to 16 C atoms, such as, by way of example and preferably, ethylamine, diethylamine, triethylamine, ethyldiisopropylamine, monoethanolamine, diethanolamine, triethanolamine, dicyclohexylamine, dimethylaminoethanol, procaine, dibenzylamine, N-methyl-morpholine, arginine, lysine, ethylenediamine and N-methylpiperidine.

Solvates for the purposes of the invention refer to those forms of the compounds of the invention which form a complex in the solid or liquid state through coordination with solvent molecules. Hydrates are a special form of solvates in which the coordination takes place with water.

For the purposes of the present invention, the substituents have the following meaning, unless otherwise specified:

Alkyl represents a linear or branched alkyl radical generally having 1 to 6, preferably 1 to 4, particularly preferably 1 to 3, carbon atoms, by way of example and preferably methyl, ethyl, n-propyl, isopropyl, tert-butyl, 2,2-dimethylprop-1-yl, n-pentyl and n-hexyl.

Heterocyclyl represents a monocyclic, heterocyclic radical having 5 or 6 ring atoms and up to 3, preferably up to 2, heteroatoms and/or hetero groups from the series N, O, S, SO, SO₂. The heterocyclyl radicals may be saturated or partly unsaturated. Preferred examples which may be mentioned are tetrahydrofuran-2-yl, tetrahydrofuran-3-yl, pyrrolidin-2-yl, pyrrolidin-3-yl, pyrrolinyl, piperidin-1-yl, piperidin-2-yl, piperidin-3-yl, piperidin-4-yl, tetrahydropyran-2-yl, tetrahydropyran-3-yl, tetrahydropyran-4-yl, piperazin-1-yl, piperazin-2-yl, morpholin-2-yl, morpholin-3-yl, morpholin-4-yl, thiomorpholin-2-yl, thiomorpholin-3-yl and thiomorpholin-4-yl.

Heteroaryl represents an aromatic, monocyclic radical having 5 or 6 ring atoms and up to 4, preferably up to 2, heteroatoms from the series S, O and N, by way of example and preferably thien-2-yl, thien-3-yl, fur-2-yl, fur-3-yl, pyrrol-1-yl, pyrrol-2-yl, pyrrol-3-yl, thiazol-2-yl, thiazol-4-yl, thiazol-5-yl, oxazol-2-yl, oxazol-4-yl, oxazol-5-yl, imidazol-1-yl, imidazol-2-yl, imidazol-4-yl, imidazol-5-yl, pyrid-2-yl, pyrid-3-yl, pyrid-4-yl, pyrimid-2-yl, pyrimid-4-yl, pyrimid-5-yl, pyrazin-2-yl, pyrazin-3-yl, pyridazin-3-yl and pyridazin-4-yl.

Halogen represents fluorine, chlorine, bromine and iodine, preferably fluorine and chlorine.

Preference is given to compounds of formula (Ia) which correspond to formula

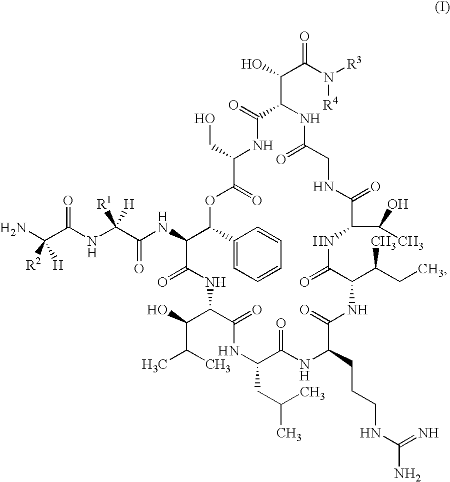

in which
R¹ represents 2-methylprop-1-yl, 2,2-dimethylprop-1-yl, 1-trimethylsilylmethyl or 3-pyridylmethyl,
   whereby 3-pyridylmethyl may be substituted with a trifluoromethyl substituent,
R² represents 2-methylprop-1-yl, 2,2-dimethylprop-1-yl, 2,2-dimethylbut-1-yl, 2-ethyl-2-methylbut-1-yl, 2,2-diethylbut-1-yl, 2,2-dimethylpent-1-yl or 1-trimethylsilylmethyl,
R³ represents C₁-C₆-alkyl,
   whereby alkyl may be substituted with 1 to 3 substituents, whereby the substituents are selected independently of one another from the group consisting of hydroxy, amino, hydroxycarbonyl, aminocarbonyl, {[bis(dimethylamino)methylene]amino}ethoxy, phenyl, 5- or 6-membered heterocyclyl, 5- or 6-membered heteroaryl, benzyloxycarbonyl and benzyloxycarbonylamino,
R⁴ represents hydrogen, C₁-C₄-alkyl, cyclopropyl or cyclopropylmethyl,
   and the salts thereof, the solvates thereof and the solvates of the salts thereof.

Preference is also given to compounds of formulae (Ia) and (I) in which
R¹ represents 2-methylprop-1-yl, 2,2-dimethylprop-1-yl, 1-trimethylsilylmethyl or 3-pyridyl-methyl,
   whereby 3-pyridylmethyl may be substituted with a trifluoromethyl substituent,
R² represents 2-methylprop-1-yl, 2,2-dimethylprop-1-yl, 2,2-dimethylbut-1-yl, 2-ethyl-2-methylbut-1-yl, 2,2-diethylbut-1-yl, 2,2-dimethylpent-1-yl or 1-trimethylsilylmethyl,
R³ represents C₁-C₄-alkyl,
   whereby alkyl may be substituted with 1 to 2 substituents, whereby the substituents are selected independently of one another from the group consisting of hydroxy, amino, hydroxycarbonyl, aminocarbonyl, {[bis(dimethylamino)methylene]amino}ethoxy, phenyl, morpholin-4-yl, pyrid-2-yl, pyrid-3-yl, pyrid-4-yl, benzyloxycarbonyl and benzyloxycarbonylamino,
R⁴ represents hydrogen or methyl,
   and the salts thereof, the solvates thereof and the solvates of the salts thereof.

Preference is also given to the compounds of formulae (Ia) and (I) in which
R¹ represents 2-methylprop-1-yl,
R² represents 2-methylprop-1-yl,
R³ represents C₁-C₃-alkyl,
   whereby alkyl may be substituted with a substituent, whereby the substituent is selected from the group consisting of hydroxy, amino, hydroxycarbonyl, aminocarbonyl, {[bis(dimethylamino)methylene]amino}ethoxy, phenyl, morpholin-4-yl, pyrid-3-yl, benzyloxycarbonyl and benzyloxycarbonylamino,
R⁴ represents hydrogen or methyl,
   and the salts thereof, the solvates thereof and the solvates of the salts thereof.

Preference is also given to the compounds of formulae (Ia) and (I) in which
R¹ represents 2,2-dimethylprop-1-yl,
R² represents 2,2-dimethylprop-1-yl,
R³ represents C₁-C₃-alkyl,
   whereby alkyl may be substituted with a substituent, whereby the substituent is selected from the group consisting of hydroxy, amino, hydroxycarbonyl, aminocarbonyl, {[bis(dimethylamino)methylene]amino}ethoxy, phenyl, morpholin-4-yl, pyrid-3-yl, benzyloxycarbonyl and benzyloxycarbonylamino,
R⁴ represents hydrogen or methyl,
   and the salts thereof, the solvates thereof and the solvates of the salts thereof.

The invention further relates to compounds which correspond to formula

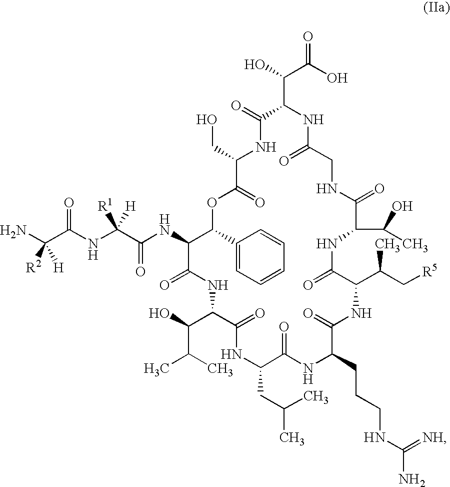

in which
R¹ represents 2-methylprop-1-yl, 2,2-dimethylprop-1-yl, 1-trimethylsilylmethyl or 3-pyridylmethyl,
   whereby 3-pyridylmethyl may be substituted with a trifluoromethyl substituent,
R¹ represents 2-methylprop-1-yl, 2,2-dimethylprop-1-yl, 2,2-dimethylbut-1-yl, 2-ethyl-2-methylbut-1-yl, 2,2-diethylbut-1-yl, 2,2-dimethylpent-1-yl or 1-trimethylsilylmethyl,
R⁵ represents hydrogen or methyl,
   and the salts thereof, the solvates thereof and the solvates of the salts thereof.

Preference is given to compounds of the formula (IIa) which correspond to formula

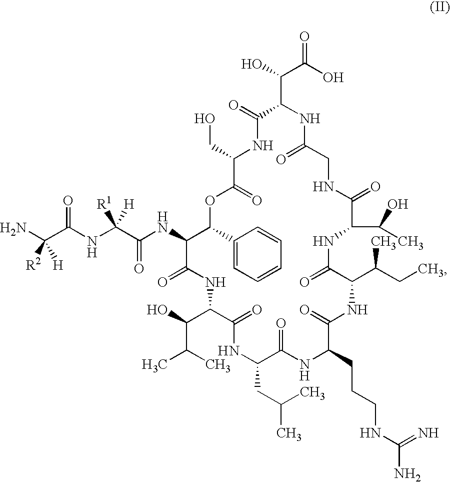

in which
R¹ represents 2-methylprop-1-yl, 2,2-dimethylprop-1-yl, 1-trimethylsilylmethyl or 3-pyridyl-methyl,
   whereby 3-pyridylmethyl may be substituted with a trifluoromethyl substituent,
R² represents 2-methylprop-1-yl, 2,2-dimethylprop-1-yl, 2,2-dimethylbut-1-yl, 2-ethyl-2-methylbut-1-yl, 2,2-diethylbut-1-yl, 2,2-dimethylpent-1-yl or 1-trimethylsilylmethyl,
   and the salts thereof, the solvates thereof and the solvates of the salts thereof.

Preference is also given to compounds of formulae (IIa) and (II) in which
R¹ represents 2-methylprop-1-yl,
R² represents 2-methylprop-1-yl,
   and the salts thereof, the solvates thereof and the solvates of the salts thereof.

Preference is also given to compounds of the formulae (Ia) and (II) in which
R¹ represents 2,2-dimethylprop-1-yl,
R² represents 2,2-dimethylprop-1-yl,
   and the salts thereof, the solvates thereof and the solvates of the salts thereof.

Preference is also given to compounds of formulae (Ia), (I), (Ia) and (II) in which R¹ represents 2-methylprop-1-yl.

Preference is also given to compounds of formulae (Ia), (I), (Ia) and (II) in which R² represents 2-methylprop-1-yl.

Preference is also given to compounds of formulae (Ia), (I), (Ia) and (II) in which R¹ and R² represent 2,2-dimethylprop-1-yl.

Preference is also given to compounds of formulae (Ia), (I), (Ia) and (II) in which R¹ represents 1-trimethylsilylmethyl and R² represents 3-pyridylmethyl.

Preference is also given to compounds of formulae (Ia) and (I) in which R⁴ represents hydrogen.

Preference is also given to compounds of formulae (Ia) and (Ia) in which R⁵ represents methyl.

The definitions of radicals indicated specifically in their respective combinations or preferred combinations of radicals are replaced irrespective of the particular combinations indicated for the radicals also as desired by the definitions of radicals of another combination.

Combinations of two or more of the abovementioned preferred ranges are very particularly preferred.

The invention further relates to a method for preparing the compounds of formula (Ia), whereby compounds of formula

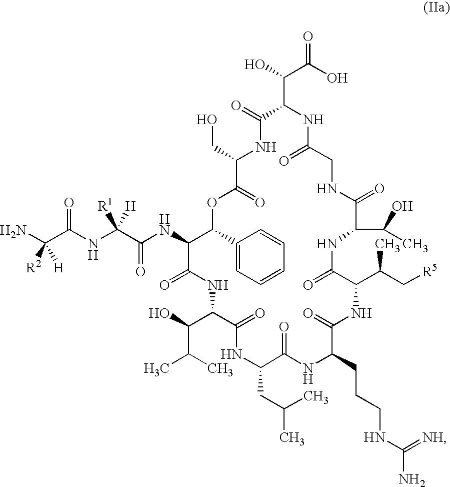

in which

R¹, R² and R⁵ have the meaning indicated above,

are reacted with compounds of formula

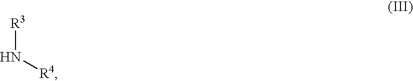

in which
R³ and R⁴ have the meaning indicated above,
The free amino group in the radical H₂N(CHR²)— is protected before the reaction according to methods known to the man of the art, for example with a Boc protecting group or a benzyloxycarbonyl protecting group, which is removed again after the reaction.

The reaction generally takes place in inert solvents, in the presence of a dehydrating reagent, where appropriate in the presence of a base, preferably in a temperature range from −30° C. to 50° C. under atmospheric pressure.

Examples of inert solvents are halohydrocarbons such as dichloromethane or trichloromethane, hydrocarbon such as benzene, nitromethane, dioxane, dimethylformamide or acetonitrile. It is likewise possible to employ mixtures of the solvents. Dichloromethane or dimethylformamide is particularly preferred.

Examples of bases are alkali metal carbonates such as, for example, sodium or potassium carbonate, or bicarbonate, or organic bases such as trialkylamines, e.g. triethylamine, N-methylmorpholine, N-methylpiperidine, 4-dimethylaminopyridine or diisopropylethylamine.

Examples of suitable dehydrating reagents in this connection are carbodiimides such as, for example, N,N′-diethyl-, N,N,′-dipropyl-, N,N′-diisopropyl-, N,N′-dicyclohexylcarbodiimide, N-(3-dimethylaminoisopropyl)-N′-ethylcarbodiimide hydrochloride (EDC), N-cyclohexyl-carbodiimide-N′-propyloxymethyl-polystyrene (PS-carbodiimide) or carbonyl compounds such as carbonyldiimidazole or 1,2-oxazolium compounds such as 2-ethyl-5-phenyl-1,2-oxazolium 3-sulfate or 2-tert-butyl-5-methylisoxazolium perchlorate, or acylamino compounds such as 2-ethoxy-1-ethoxycarbonyl-1,2-dihydroquinoline, or propanephosphonic anhydride, or isobutyl chloroformate, or bis(2-oxo-3-oxazolidinyl)phosphoryl chloride, or O-(benzotriazol-1-yl)-N,N,N′,N′-tetramethyluronium hexafluorophosphate (HBTU), 2-(2-oxo-1-(2H)-pyridyl)-1,1,3,3-tetramethyluronium tetrafluoroborate (TPTU) or O-(7-azabenzotriazol-1-yl)-N,N,N′,N′-tetramethyluronium hexafluorophosphate (HATU), or 1-hydroxybenzotriazole (HOBt), or benzotriazol-1-yloxytris(dimethylamino)phosphonium hexafluorophosphate (BOP), or benzotriazol-1-yloxytris(pyrrolidino)phosphonium hexafluorophosphate (PyBOP), or N-hydroxy-succinimide, or mixtures thereof, with bases.

The condensation is preferably carried out with HATU and N-methylmorpholine.

Preferred methods are those in which R⁵ represents methyl.

Preferred methods are also those in which
R¹ represents 2-methylprop-1-yl, 2,2-dimethylprop-1-yl, 1-trimethylsilylmethyl or 3-pyridyl-methyl,
   whereby 3-pyridylmethyl may be substituted with a trifluoromethyl substituent,
R² represents 2-methylprop-1-yl, 2,2-dimethylprop-1-yl, 2,2-dimethylbut-1-yl, 2-ethyl-2-methylbut-1-yl, 2,2-diethylbut-1-yl, 2,2-dimethylpent-1-yl or 1-trimethylsilylmethyl,
R³ represents C₁-C₄-alkyl,
   whereby alkyl may be substituted with 1 to 2 substituents, whereby the substituents are selected independently of one another from the group consisting of hydroxy, amino, hydroxycarbonyl, aminocarbonyl, {[bis(dimethylamino)methylene]amino}ethoxy, phenyl, morpholin-4-yl, pyrid-2-yl, pyrid-3-yl, pyrid-4-yl, benzyloxycarbonyl and benzyloxycarbonylamino,
R⁴ represents hydrogen or methyl.

Preferred methods are also those in which
R¹ represents 2-methylprop-1-yl,
R² represents 2-methylprop-1-yl,
R³ represents C₁-C₃-alkyl,
   whereby alkyl may be substituted with a substituent, whereby the substituent is selected from the group consisting of hydroxy, amino, hydroxycarbonyl, aminocarbonyl, {[bis(dimethylamino)methylene]amino}ethoxy, phenyl, morpholin-4-yl, pyrid-3-yl, benzyloxycarbonyl and benzyloxycarbonylamino,
R⁴ represents hydrogen or methyl.

Preferred methods are also those in which
R¹ represents 2,2-dimethylprop-1-yl,
R² represents 2,2-dimethylprop-1-yl,
R³ represents C₁-C₃-alkyl,
   whereby alkyl may be substituted with a substituent, whereby the substituent is selected from the group consisting of hydroxy, amino, hydroxycarbonyl, aminocarbonyl, {[bis(dimethylamino)methylene]amino}ethoxy, phenyl, morpholin-4-yl, pyrid-3-yl, benzyloxycarbonyl and benzyloxycarbonylamino,
R⁴ represents hydrogen or methyl.

The compounds of formulae (Ia), (I), (IIa) and (II) which are in the form of salts can be converted for example by reaction with hydrochloric acid or methanesulfonic acid into a salt with a different counter ion.

The compounds of formulae (Ia), (I), (IIa) and (II) which are in the form of salts can be converted into the free base by reaction with a base.

The compounds of formula (III) are known or can be synthesized by known methods from the corresponding starting materials.

It has surprisingly been found that compounds of formula (Ia) can be prepared by selective hydrolysis of compounds of formula

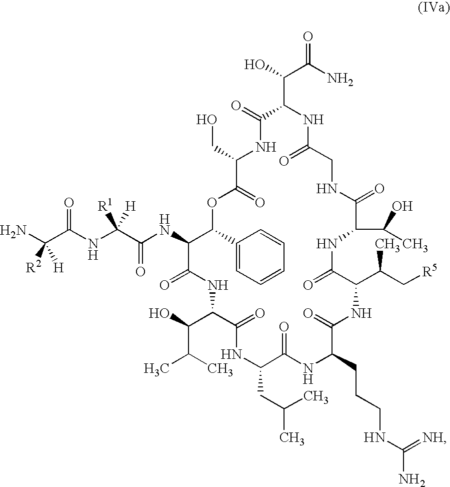

in which
R¹, R² and R⁵ have the meaning indicated above,
with an acid in a suitable solvent.

The reaction generally takes place in a solvent, preferably in a temperature range from room temperature to 50° C. under atmospheric pressure.

Examples of solvents are mixtures of dioxane with water or tetrahydrofuran with water.

Examples of acids are mineral acids such as hydrochloric acid or other strong acids such as methanesulfonic acid; hydrochloric acid is preferred.

The invention further relates to a method for preparing the compounds of formula (IIa) by hydrolysis of a compound of formula (IVa) with an acid in a suitable solvent.

The method is preferably carried out in a temperature range from room temperature to 50° C. under atmospheric pressure.

The method is particularly preferably carried out at room temperature under atmospheric pressure.

The solvent is preferably selected from the group consisting of dioxane, tetrahydrofuran, water and mixtures thereof.

The solvent is particularly preferably selected from the group consisting of a mixture of dioxane with water and a mixture of tetrahydrofuran with water.

The acid is preferably selected from the group consisting of the mineral acids and other strong acids; the acid is in particular a mineral acid.

The acid is preferably selected from the group consisting of hydrochloric acid and methanesulfonic acid.

The acid is particularly preferably hydrochloric acid.

Preferred methods are those in which R⁵ represents methyl.

Preferred methods are also those in which R¹ and R² represent 2-methylprop-1-yl.

Preferred methods are also those in which R¹ and R² represent 2,2-dimethylprop-1-yl.

The compounds of formula (IVa) are known or can be prepared by reacting compounds of formula

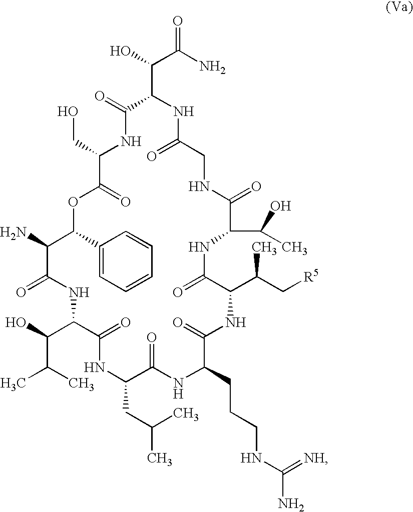

in which R⁵ has the meaning indicated above,
with compounds of formula

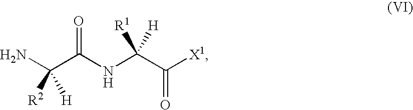

in which
R¹ and R² have the meaning indicated above, and
X¹ represents halogen, preferably bromine, chlorine or fluorine, or hydroxy.

If X¹ represents halogen, the reaction generally takes place in inert solvents, where appropriate in the presence of a base, preferably in a temperature range from −30° C. to 50° C. under atmospheric pressure.

Examples of inert solvents are tetrahydrofuran, methylene chloride, pyridine, dioxane or dimethylformamide. Tetrahydrofuran or methylene chloride are preferred as inert solvents.

Examples of bases are triethylamine, diisopropylethylamine or N-methylmorpholine; diisopropylethylamine is preferred.

If X¹ represents hydroxy, the reaction generally takes place in inert solvents, in the presence of a dehydrating reagent, where appropriate in the presence of a base, preferably in a temperature range from −30° C. to 50° C. under atmospheric pressure.

Examples of inert solvents are halohydrocarbons such as dichloromethane or trichloromethane, hydrocarbon such as benzene, nitromethane, dioxane, dimethylformamide or acetonitrile. It is likewise possible to employ mixtures of the solvents. Dichloromethane or dimethylformamide is particularly preferred.

Examples of suitable dehydrating reagents in this connection are carbodiimides such as, for example, N,N′-diethyl-, N,N,′-dipropyl-, N,N′-diisopropyl-, N,N′-dicyclohexylcarbodiimide, N-(3-dimethylaminoisopropyl)-N′-ethylcarbodiimide hydrochloride (EDC), N-cyclohexyl-carbodiimide-N′-propyloxymethyl-polystyrene (PS-carbodiimide) or carbonyl compounds such as carbonyldiimidazole or 1,2-oxazolium compounds such as 2-ethyl-5-phenyl-1,2-oxazolium 3-sulfate or 2-tert-butyl-5-methylisoxazolium perchlorate, or acylamino compounds such as 2-ethoxy-1-ethoxycarbonyl-1,2-dihydroquinoline, or propanephosphonic anhydride, or isobutyl chloroformate, or bis(2-oxo-3-oxazolidinyl)phosphoryl chloride, or benzotriazolyloxytri(dimethyl-amino)phosphonium hexafluorophosphate, or O-(benzotriazol-1-yl)-N,N,N′,N′-tetramethyluronium hexafluorophosphate (HBTU), 2-(2-oxo-1-(2H)-pyridyl)-1,1,3,3-tetramethyluronium tetrafluoroborate (TPTU) or O-(7-azabenzotriazol-1-yl)-N,N,N′,N′-tetramethyluronium hexafluorophosphate (HATU), or 1-hydroxybenzotriazole (HOBt), or benzotriazol-1-yloxytris(dimethylamino)phosphonium hexafluorophosphate (BOP) or N-hydroxysuccinimide, or mixtures thereof, with bases.

Examples of bases are alkali metal carbonates such as, for example, sodium or potassium carbonate, or bicarbonate, or organic bases such as trialkylamines, e.g. triethylamine, N-methylmorpholine, N-methylpiperidine, 4-dimethylaminopyridine or diisopropylethylamine.

The condensation is preferably carried out with HATU or with EDC in the presence of HOBt.

The compounds of formula (VI) carry protecting groups where appropriate, so that in these cases the reaction of the compound of formula (Va) with compounds of formula (VI) is followed by a removal of the protecting groups using, for example, trifluoroacetic acid by methods known to the man of the art.

The compounds of formula (Va) can be synthesized by double Edmann degradation from lysobactin (Example 1A) or katanosin A.

The compounds of formula (VI) are known or can be synthesized by known methods from the appropriate starting materials.

The preparation of the compounds of the invention can be illustrated by the following synthesis scheme.

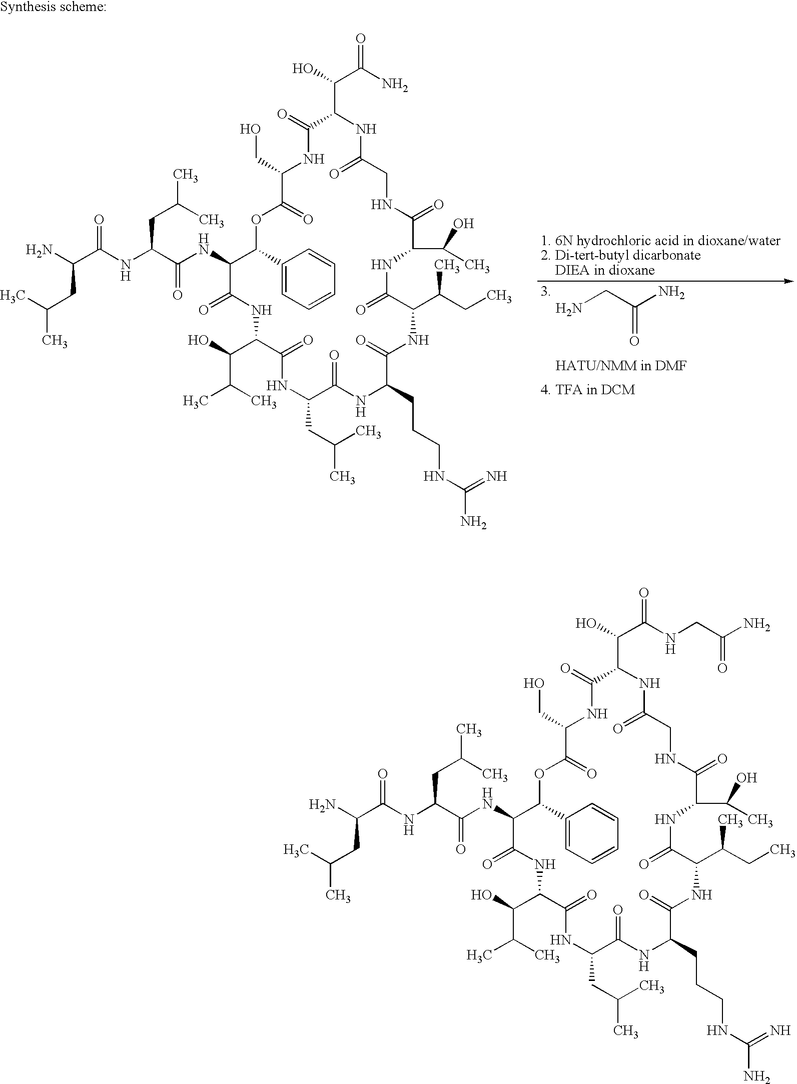

The compounds of the invention of formula (Ia) and (I) show a valuable range of pharmacological effects which could not have been predicted. They show an antibacterial activity.

They are therefore suitable for use as medicaments for the treatment and/or prophylaxis of diseases in humans and animals.

The compounds of the invention of formulae (Ia) and (I) are distinguished by a higher free fraction (fᵤ) in rat and human plasma compared with lysobactin.

The compounds of the invention of formulae (Ia) and (I) are distinguished by a lower nephrotoxicity compared with lysobactin.

The compounds of the invention of formulae (Ia) and (I) are distinguished by a better solubility compared with lysobactin.

The described nonadepsipeptides act as inhibitors of the bacterial cell wall biosynthesis.

The preparations of the invention are particularly effective against bacteria and bacteroid microorganisms. They are therefore particularly suitable for the prophylaxis and chemotherapy of local and systemic infections caused by these pathogens in human and veterinary medicine.

The preparations of the invention can in principle be used against all bacteria and bacteroid microorganisms possessing a bacterial cell wall (murein sacculus) and the corresponding enzyme systems, for example against the following pathogens or mixtures of the following pathogens:

Gram-negative cocci (Neisseria gonorrhoeae) as well as Gram-negative rods such as enterobacteriaceae, e.g. Escherichia coli, Haemophilus influenzae, Pseudomonas, Klebsiella, Citrobacter (C. freundii, C. divernis), Salmonella and Shigella; furthermore Enterobacter (E. aerogenes, E. agglomerans), Hafnia, Serratia (S. marcescens), Providencia, Yersinia, as well as the genus Acinetobacter, Branhamella and Chlamydia. The antibacterial range additionally includes strictly anaerobic bacteria such as, for example, Bacteroides fragilis, representatives of the genus Peptococcus, Peptostreptococcus, as well as the genus Clostridium; furthermore mycobacteria, e.g. M. tuberculosis. The compounds of the invention show a particularly pronounced activity against Gram-positive cocci, e.g. staphylococci (S. aureus, S. epidermidis, S. haemolyticus, S. carnosus), enterococci (E. faecalis, E. faecium) and streptococci (S. agalactiae, S. pneumoniae, S. pyogenes).

The above list of pathogens is merely by way of example and is by no means to be interpreted restrictively. Examples which may be mentioned of diseases which are caused by the pathogens mentioned or mixed infections and can be prevented, improved or healed by the preparations of the invention are:

Infectious diseases in humans such as, for example, uncomplicated and complicated urinary tract infections, uncomplicated cutaneous and superficial infections, complicated cutaneous and soft tissue infections, hospital-acquired and community-acquired pneumonia, nosocomial pneumonias, acute exacerbations and secondary bacterial infections of chronic bronchitis, acute otitis media, acute sinusitis, streptococcal pharyngitis, bacterial meningitis, uncomplicated gonococcal and non-gonococcal urethritis/cervicitis, acute prostatitis, endocarditis, uncomplicated and complicated intra-abdominal infections, gynecological infections, pelvic inflammatory disease, bacterial vaginosis, acute and chronic osteomyelitis, acute bacterial arthritis, empirical therapy in febrile neutropenic patients, furthermore bacteremias, MRSA infections, acute infectious diarrhea, Helicobacter pylori infections, postoperative infections, odontogenic infections, opthalmological infections, postoperative infections (including periproctal abscess, wound infections, biliary infections, mastitis and acute appendicitis), cystic fibrosis and bronchiectasis.

Apart from humans, bacterial infections can also be treated in other species. Examples which may be mentioned are:

Pigs: diarrhea, enterotoxemia, sepsis, dysentery, salmonellosis, metritis-mastitis-agalactiae syndrome, mastitis;

Ruminants (cattle, sheep, goats): diarrhea, sepsis, bronchopneumonia, salmonellosis, pasteurellosis, genital infections;

Horses: bronchopneumonias, joint ill, puerperal and postpuerperal infections, salmonellosis;

Dogs and cats: bronchopneumonia, diarrhea, dermatitis, otitis, urinary tract infections, prostatitis;

Poultry (chicken, turkeys, quail, pigeons, ornamental birds and others): E. coli infections, chronic airway diseases, salmonellosis, pasteurellosis, psittacosis.

It is likewise possible to treat bacterial diseases in the rearing and management of productive and ornamental fish, in which case the antibacterial spectrum is extended beyond the pathogens mentioned above to further pathogens such as, for example, Pasteurella, Brucella, Campylobacter, Listeria, Erysipelothris, corynebacteria, Borellia, Treponema, Nocardia, Rikettsia, Yersinia.

The compounds of the invention of formulae (IIa) and (II) form important intermediates in the synthesis of the compounds of formulae (Ia) and (I).

The present invention further relates to the use of the compounds of the invention of formulae (Ia) and (I) for the treatment and/or prophylaxis of diseases, especially of bacterial infectious diseases.

The present invention further relates to the use of the compounds of the invention of formulae (Ia) and (I) for the treatment and/or prophylaxis of diseases, especially of the aforementioned diseases.

The present invention further relates to the use of the compounds of the invention of formulae (Ia) and (I) for manufacturing a medicament for the treatment and/or prophylaxis of diseases, especially of the aforementioned diseases.

The compounds of the invention of formulae (Ia) and (I) are preferably used to manufacture medicaments suitable for the prophylaxis and/or treatment of bacterial diseases.

The present invention further relates to a method for the treatment and/or prophylaxis of diseases, especially of the aforementioned diseases, using an antibacterially effective amount of the compounds of the invention of formulae (Ia) and (I).

The present invention further relates to medicaments comprising at least one compound of the invention of formulae (Ia) and (I) and at least one or more further active ingredients, in particular for the treatment and/or prophylaxis of the aforementioned diseases. Preferred active ingredients for combination are compounds having antibacterial activity and having a different range of effects, in particular a supplementary range of effects, and/or being synergistic to the compounds of the invention.

The compounds of the invention of formulae (Ia) and (I) can act systemically and/or locally. For this purpose, they can be administered in a suitable way such as, for example, orally, parenterally, pulmonarily, nasally, sublingually, lingually, buccally, rectally, dermally, transdermally, conjunctivaly or otically, or as an implant or stent.

The compounds of the invention can be administered in administration forms suitable for these administration routes.

Suitable for oral administration are administration forms which function according to the prior art and deliver the compounds of the invention rapidly and/or in modified fashion, and which contain the compounds of the invention in crystalline and/or amorphized and/or dissolved form, such as, for example, tablets (uncoated or coated tablets, for example having enteric coatings or coatings which or dissolve with a delay or are insoluble and control the release of the compound of the invention), tablets or films/wafers, which disintegrate rapidly in the oral cavity, films/lyophilizates, capsules (for example hard or soft gelatin capsules), sugar-coated tablets, granules, pellets, powders, emulsions, suspensions, aerosols or solutions.

Parenteral administration can take place with avoidance of an absorption step (e.g. intravenous, intraarterial, intracardiac, intraspinal or intralumbar) or with inclusion of an absorption (e.g. intramuscular, subcutaneous, intracutaneous, percutaneous or intraperitoneal). Administration forms suitable for parenteral administration are, inter alia, preparations for injection and infusion in the form of solutions, suspensions, emulsions, lyophilizates or sterile powders.

Suitable for the other administration routes are, for example, pharmaceutical forms for inhalation (inter alia powder inhalers, nebulizers), nasal drops, solutions, sprays; tablets, films/wafers or capsules, for lingual, sublingual or buccal administration, suppositories, preparations for the ears or eyes, vaginal capsules, aqueous suspensions (lotions, shaking mixtures), lipophilic suspensions, ointments, creams, transdermal therapeutic systems (such as, for example, patches), milk, pastes, foams, dusting powders, implants or stents.

The compounds of the invention of formulae (Ia) and (I) can be converted into the stated administration forms. This can take place in a manner known per se by mixing with inert, nontoxic, pharmaceutically acceptable excipients. These excipients include, inter alia, carriers (for example microcrystalline cellulose, lactose, mannitol), solvents (e.g. liquid polyethylene glycols), emulsifiers and dispersants or wetting agents (for example sodium dodecyl sulfate, polyoxysorbitan oleate), binders (for example polyvinylpyrrolidone), synthetic and natural polymers (for example albumin), stabilizers (e.g. antioxidants such as, for example, ascorbic acid), colors (e.g. inorganic pigments such as, for example, iron oxides) and taste and/or odor corrigents.

The present invention further relates to medicaments which comprise at least one compound of the invention of formulae (Ia) and (I), usually together with one or more inert, nontoxic, pharmaceutically acceptable excipients, and to the use thereof for the aforementioned purposes.

It has generally proved advantageous to administer on intravenous administration amounts of about 0.001 to 100 mg/kg, preferably about 0.1 to 10 mg/kg of body weight to achieve effective results, and on oral administration the dosage is about 0.01 to 50 mg/kg, preferably 0.5 to 10 mg/kg, of body weight.

It may nevertheless be necessary where appropriate to deviate from the stated amounts, in particular as a function of the body weight, route of administration, individual response to the active ingredient, nature of the preparation and time or interval over which administration takes place. Thus, it may be sufficient in some cases to make do with less than the aforementioned minimum amount, whereas in other cases the stated upper limit must be exceeded. It may in the event of an administration of larger amounts be advisable to divide these into a plurality of individual doses over the day.

The percentage data in the following tests and examples are, unless indicated otherwise, percentages by weight; parts are parts by weight. Solvent ratios, dilution ratios and concentration data for the liquid/liquid solutions are in each case based on volume.

### DETAILED DESCRIPTION OF PREFERRED EMBODIMENTS

### A. Examples

### Abbreviations

Area (peak) area
BHI brain heart infusion
Boc tert-butyloxycarbonyl
br. broad signal (in NMR spectra)
calc. calculated
conc. concentrated
d doublet (in NMR spectra)
DCI direct chemical ionization (in MS)
DCM dichloromethane
DIEA N,N-diisopropylethylamine
DMSO dimethyl sulfoxide
DMF N,N-dimethylformamide
EA ethyl acetate (acetic acid ethyl ester)
EDC 1-ethyl-3-(3-dimethylaminopropyl)carbodiimide (also EDCI)
EDC×HCl 1-ethyl-3-(3-dimethylaminopropyl)carbodiimide hydrochloride
EI electron impact ionization (in MS)
ESI electrospray ionization (in MS)
Ex. Example
h hour
HATU O-(7-azabenzotriazol-1-yl)-N,N,N′,N′-tetramethyluronium hexa-fluorophosphate
HOBt 1-hydroxybenzotriazole
HPLC high pressure, high performance liquid chromatography
HR high resolution
i. v. in vacuo
LC-MS coupled liquid chromatography-mass spectroscopy
LDA lithium diisopropylamide
m middle (in UV and IR spectra)
m multiplet (in NMR spectra)
MALDI matrix-assisted laser desorption/ionization
MIC minimum inhibitory concentration
min minute(s)
m.p. melting point
MRSA methicillin-resistant Staphylococcus aureus
MS mass spectroscopy
NCCLS National Committee for Clinical Laboratory Standards
neg. negative
NMM N-methylmorpholine
NMR nuclear magnetic resonance spectroscopy
p.a. pro analysi
Pd—C palladium on carbon
pos. positive
quant. quantitative
RP-HPLC reverse phase HPLC
RT room temperature
Rₜ retention time (in HPLC)
strong (in UV and IR spectra)
singlet (in NMR spectra)
sat. saturated
TBTU O-(benzotriazol-1-yl)-N,N,N′,N′-tetramethyluronium tetrafluoroborate
TCTU O-(1H-6-chlorobenzotriazole-1-yl)-1,1,3,3-tetramethyluronium tetrafluoroborate
TFA trifluoroacetic acid
TFE 2,2,2-trifluoroethanol
THF tetrahydrofuran
TLC thin-layer chromatography
TOF time of flight
UV ultraviolet
Vis visible
VRSA vancomycin-resistant Staphylococcus aureus
w weak (in UV and IR spectra)
Z, Cbz benzyloxycarbonyl

### REFERENCES

Concerning the nomenclature of peptides and cyclodepsipeptides, compare:
1. A Guide to IUPAC Nomenclature of Organic Compounds (Recommendations 1993), 1993, Blackwell Scientific publications.
2. Nomenclature and symbolism for amino acids and peptides. Recommendations 1983. IUPAC-IUB Joint Commission on Biochemical Nomenclature, UK. Biochemical Journal 1984, 219, 345-373, and cited literature.
3. For the nomenclature of nonadepsipeptide derivatives which are derivatized in the amino acid side chains, the IUPAC prefix system is used for addressing the respective derivatization site (IUPAC, Nomenclature and Symbolism for Amino Acids and Peptides, Names and Symbols for Derivatives of Named Peptides, Section 3AA-22, Recommendations 1983-1992). Thus, for example, N^{ω,6}-acetyllysobactin refers to a lysobactin acetylated on amino acid 6 (calculated from the N terminus of the depsipeptide, i.e. here D-Arg) specifically on the terminal nitrogen atom. Analogously, O^{3.11}-methyllysobactin refers to a derivative methylated on amino acid 11 (Ser) on the side-chain oxygen atom (O³).

General LC-MS, HR-MS, HPLC and Gel Chromatography Methods

Method 1 (HPLC): HPLC instrument type: HP 1100 series; UV DAD column: Zorbax Eclipse XBD-C8 (Agilent), 150 mm×4.6 mm, 5 μm; eluent A: 5 ml of HClO₄/l of water, eluent B: acetonitrile; gradient: 0-1 min 10% B, 1-4 min 10-90% B, 4-5 min 90% B; flow rate: 2.0 ml/min; oven: 30° C.; UV detection: 210 and 254 nm.

Method 2 (HPLC): Column: Kromasil RP-18, 60 mm×2 mm, 3.5 μm; eluent A: 5 ml of HClO₄/l of water, eluent B: acetonitrile; gradient: 0 min 2% B, 0.5 min 2% B, 4.5 min 90% B, 9 min 90% B; flow rate: 0.75 ml/min; oven: 30° C.; UV detection: 210 nm.

Method 3 (LC-MS): MS instrument type: Micromass ZQ; HPLC instrument type: HP 1100 Series; UV DAD; column: Phenomenex Synergi 2μ Hydro-RP Mercury 20 mm×4 mm; eluent A: 1 l of water+0.5 ml of 50% formic acid, eluent B: 1 l of acetonitrile+0.5 ml of 50% formic acid; gradient: 0.0 min 90% A→2.5 min 30% A→3.0 min 5% A→4.5 min 5% A; flow rate: 0.0 min 1 ml/min, 2.5 min/3.0 min/4.5 min. 2 ml/min; oven: 50° C.; UV detection: 210 nm.

Method 4 (HPLC): column: Kromasil RP-18, 250 mm×4 mm, 5 μm; eluent A: 5 ml of HClO₄/l of water, eluent B: acetonitrile; gradient: 0 min 5% B, 10 min 95% B; flow rate: 1 ml/min; oven: 40° C.; UV detection: 210 nm.

Method 5 (HPLC): column: Kromasil RP-18, 250 mm×4 mm, 5 μm; eluent A: 2 ml of HClO₄/l of water, eluent B: acetonitrile; isocratic: 45% B, 55% A; flow rate: 1 ml/min; oven: 40° C.; UV detection: 210 nm.

Method 6 (preparative HPLC): Instrument: Gilson Abimed HPLC; binary pump system; column: Nucleodur C₁₈ Gravity, Macherey-Nagel, 5 μm; 250 mm×21 mm; eluent A: water/0.05-0.1% trifluoroacetic acid, eluent B: acetonitrile/0.05-0.1% trifluoroacetic acid; gradient: 0-8 min 5% B, 8-40 min 5-60% B, 40-60 min 60% B, 60-75 min 60-100% B, 75-80 min 100% B, then regeneration of the chromatography column; flow rate: 7-15 ml/min; UV detection: 210 nm.

Method 7 (preparative HPLC): Instrument: Gilson Abimed HPLC; binary pump system; column: Kromasil-100A C₁₈, 5 μm; 250 mm×30 mm; eluent A: water/0.05-0.5% TFA, eluent B: acetonitrile; gradient: 0-5 min 5% B, 5.01-10 min 10% B, 10.01-20 min 40% B, 20.01-27 min 50% B, 27.01-40 min 60% B, 40.01-45 min 90% B, 45.01-60 min 100% B; flow rate: 15-60 ml/min; UV detector 210 nm.

Method 8 (preparative HPLC): Instrument: Gilson Abimed HPLC; binary pump system; column: Kromasil-100A C₁₈, 5 μm; 250 mm×30 mm; eluent A: water/0.05-0.5% TFA, eluent B: acetonitrile; 0-10 min 10% B, ramp, 10.01-55 min 100% B; flow rate: 30 ml/min; UV detector 210 nm.

Method 9 (Sephadex LH-20 gel chromatography): Gel chromatography is carried out without pressure on Sephadex LH-20 (Pharmacia). Fractions are taken according to the UV activity (UV detector for 210 nm, Knauer) (ISCO Foxy 200 fraction collector). Column dimensions: 60×21 cm (2500-5000 μmol scale); 50×10 cm (500-2500 μmol scale); 30×5 cm (250-500 μmol scale); 25×4 cm (50-250 μmol scale); 40×2 cm (5-50 μmol scale).

Method 10 (LC-MS): MS instrument type: Micromass ZQ; HPLC instrument type: Waters Alliance 2795/HP 1100; column: Phenomenex Synergi 2 p Hydro-RP Mercury 20 mm×4 mm; eluent A: 1 l of water+0.5 ml of 50% formic acid, eluent B: 1 l of acetonitrile+0.5 ml of 50% formic acid; gradient: 0.0 min 90% A→2.5 min 30% A→3.0 min 5% A→4.5 min 5% A; flow rate: 0.0 min 1 ml/min, 2.5 min/3.0 min/4.5 min 2 ml/min; oven: 50° C.; UV detection: 210 nm.

Method 11 (TOF-HR-MS): TOF-HR-MS-ESI+ spectra are recorded using a Micromass LCT instrument (capillary voltage: 3.2 KV, cone voltage: 42 V, source temperature: 120° C., desolvation temperature: 280° C.). A syringe pump (Harvard Apparatus) is hereby used for supplying the sample. Leucine-encephalin (Tyr-Gly-Gly-Phe-Leu) is used as standard.

Method 12 (HPLC): column: Gilson Abimed HPLC; Varian binary pump system; Phenomenex Luna C18 5μ 250 mm×20 mm; flow rate: 25 ml/min; oven: RT; UV detection: 210 nm; eluent A: water/0.2% TFA, eluent B: acetonitrile; isocratic 50% B.

Method 13 (HPLC): column: Gilson Abimed HPLC; Varian binary pump system; Kromasil 100 C18 5μ 250 mm×20 mm; flow rate: 25 ml/min; oven: RT; UV detection: 210 nm; eluent A: water/0.2% TFA, eluent B: acetonitrile; isocratic 65% B.

Method 14 (analytical HPLC): HPLC instrument type: HP 1100 Series; UV DAD; column: Phenomenex Synergi 2 p Hydro-RP Mercury 20 mm×4 mm; eluent A: 1 l of water+0.5 ml of 50% formic acid, eluent B: 1 l of acetonitrile+0.5 ml of 50% formic acid; gradient: 0.0 min 90% A→2.5 min 30% A→3.0 min 5% A→4.5 min 5% A; flow rate: 0.0 min 1 ml/min, 2.5 min/3.0 min/4.5 min. 2 ml/min; oven: 50° C.; UV detection: 210 nm.

Method 15 (analytical HPLC): HPLC instrument type: HP 1050 Series; UV DAD; column: Zorbax 300 mSB-C18 3.5μ, 4.6 mm×150 mm; eluent A: 1 l of water+0.1% trifluoroacetic acid, eluent B: 400 ml of acetonitrile/600 ml of water+0.1% trifluoroacetic acid; gradient: 0.0 min 100% A, 1.3 min 10% B, 18.0 min 80% B, 20.0 min 80% B, 21.0 min 100% B, 25.0 min 100% B, 26.0 min 0% B, 30.0 min 0% B, flow rate: 1 ml/min; oven: 40° C.; UV detection: 210 nm.

Method 16 (analytical HPLC): HPLC instrument type: HP 1050 Series; UV DAD; column: Zorbax 300 mSB-C18 3.5μ, 4.6 mm×150 mm; eluent A: 1 l of water+0.1% trifluoroacetic acid, eluent B: 400 ml of acetonitrile/600 ml of water+0.1% trifluoroacetic acid; gradient: 0.0 min 100% A, 2.0 min 10% B, 50.0 min 80% B, 52.0 min 100% B, 55.0 min 100% A, 60.0 min 100% A. flow rate: 1 ml/min; oven: 40° C.; UV detection: 210 nm.

General Procedures

General Procedure 1 (Removal of Boc Protecting Groups Using TFA)

The Boc-protected compound (2-15 μmol) is suspended in dichloromethane (1 ml) and then, under an argon protective gas atmosphere, trifluoroacetic acid (3 ml) in dichloromethane (10 ml) is added, and the mixture is stirred at RT until the HPLC chromatogram shows complete conversion (e.g. Method 14). The solvent is then distilled off in vacuo, during which the bath temperature should not exceed 30° C. The crude product is suspended in toluene, again concentrated on a rotary evaporator and dried under high vacuum. This procedure is repeated several times (2-5×).

General Procedure 2 (Hydrogenolytic Ester Cleavage/Carbamide Cleavage)

The peptidic benzyl ester (1-15 μmol) is dissolved in dioxane (2 ml) and 0.1% aqueous trifluoroacetic acid (3 ml) and, under an argon protective gas atmosphere, 10% palladium/carbon (10 mol %) is added. Hydrogenation is carried out at RT under atmospheric pressure until analytical HPLC (e.g. Method 14) shows complete conversion (about 30 min). The reaction mixture is filtered (e.g. through a syringe filter, kieselguhr, Celite®) and finepurified by preparative RP-HPLC.

General Procedure 3 (Amide Coupling)

Under an argon protective gas atmosphere, firstly HATU (5-15 equivalents) and then NMM (5-20 equivalents) are added to a solution of the carboxylic acid cyclopeptide (1.0 equivalent) and the amine (5-15 equivalents) in dry DMF (5-30 μmol/ml) at 0° C. The reaction mixture is slowly warmed to RT and is stirred at this temperature until complete conversion is evident. The reaction mixture is evaporated under high vacuum and purified by chromatography.

Starting Compounds

### Example 1A

D-Leucyl-N¹-{(3S,6S,12S,15S,18R,21S,24S,27S,28R)-6-[(1S)-2-amino-1-hydroxy-2-oxo-ethyl]-18-(3-{[amino(imino)methyl]amino}propyl)-12-[(1S)-1-hydroxyethyl]-3-(hydroxy-methyl)-24-[(1R)-1-hydroxy-2-methylpropyl]-21-isobutyl-15-[(1S)-1-methylpropyl]-2,5,8,11,14,17,20,23,26-nonaoxo-28-phenyl-1-oxa-4,7,10,13,16,19,22,25-octaazacyclo-octacosan-27-yl}-L-leucinamide Bistrifluoroacetate (Lysobactin)

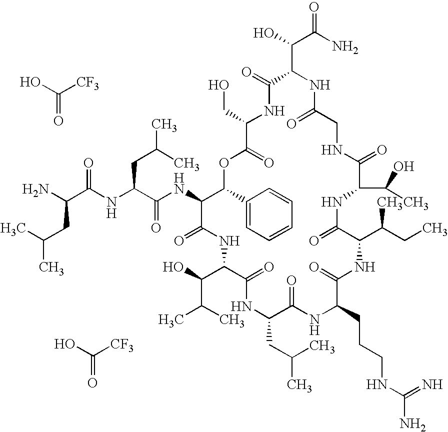

Fermentation:

Culture Medium:

YM: yeast-malt agar: D-glucose (4 g/l), yeast extract (4 g/l), malt extract (10 g/l), 1 liter of Lewatit water. The pH is adjusted to 7.2 before the sterilization (20 minutes at 121° C.).

HPM: mannitol (5.4 g/l), yeast extract (5 g/l), meat peptone (3 g/l).

Working cell bank: the lyophilized strain (ATCC 53042) is grown in 50 ml of YM medium.

Flask fermentation: 150 ml of YM medium or 100 ml of HPM medium in a 1 l Erlenmeyer flask are inoculated with 2 ml of the working cell bank and left to grow at 28° C. on a shaker at 240 rpm for 30-48 hours.

30 l Fermentation: 300 ml of the flask fermentation (HPM medium) are used to inoculate a sterile 30 l nutrient medium solution (1 ml of antifoam SAG 5693/1). This culture is left to grow at 28° C., 300 rpm aerating with sterile air at 0.3 vvm for 21 hours. The pH is kept constant at pH=7.2 using 1M hydrochloric acid. In total, 880 ml of 1M hydrochloric acid are added during the culturing time.

Main culture (200 l): 15×150 ml of YM medium in 1 l Erlenmeyer flasks are inoculated with 2 ml of the working cell bank and left to grow at 28° C. and 240 rpm on a shaker for 48 hours. 2250 ml of this culture are used to inoculate a sterile 200 l nutrient medium solution (YM) (1 ml of antifoam SAG 5693/1) and left to grow at 28° C., 150 rpm aerating with sterile air at 0.3 vvm for 18.5 hours.

Hourly Samples (50 ml) are taken to check the progress of the fermentation. 2 ml of this culture broth are mixed with 1 ml of methanol (0.5% trifluoroacetic acid) and filtered through a 0.45 μm filter. 30 μl of this suspension are analyzed by HPLC (Method 1 and Method 2).

After 18.5 hours, the culture broth of the main culture is separated into supernatant and sediment at 17 000 rpm.

Isolation:

The supernatant (183 l) is adjusted to pH 6.5-7 using concentrated trifluoroacetic acid or a sodium hydroxide solution and loaded onto a Lewapol column (OC 1064, 60 l contents). Elution is then carried out with pure water, water/methanol 1:1 and then with pure methanol (with 0.1% trifluoroacetic acid). This organic phase is concentrated in vacuo to a remaining aqueous residue of 11.5 l.

The remaining aqueous phase is bound to silica gel C₁₈ and fractionated (MPLC, Biotage Flash 75, 75×30 cm, KP-C18-WP, 15-20 μm, flow rate: 30 ml; eluent: acetonitrile/water with 0.1% trifluoroacetic acid; gradient: 10%, 15% and 40% acetonitrile). The 40% acetonitrile phase, which contains the major amount of example 1A, is concentrated in vacuo and then lyophilized (˜13 g). This mixture of solids is separated in 1.2 g portions initially on a preparative HPLC (Method 3), then by gel filtration on Sephadex LH-20 (5×70 cm, acetonitrile/water 1:1, in each case with 0.05% trifluoroacetic acid) and a further preparative HPLC (Method 4).

This process yields 2250 mg of example 1A.

The sediment is taken up in 4 l of 4:1 acetone/water, mixed with 2 kg of Celite, adjusted to pH=6 using trifluoroacetic acid, stirred and centrifuged. The solvent is concentrated in vacuo, and the residue is freeze dried. The resulting lyophilizate (89.9 g) is taken up in methanol, filtered, concentrated and separated on silica gel (Method 5). Example 1A is then purified by gel filtration (Sephadex LH-20, 5×68 cm, water/acetonitrile 9:1 (with 0.05% trifluoroacetic acid), flow rate: 2.7 ml/min, fraction size 13.5 ml) to give the pure substance.

This process yields 447 mg of example 1A.

HPLC (Method 1): Rₜ=6.19 min

MS (ESIpos): m/z=1277 (M+H)⁺

¹H NMR (500.13 MHz, d₆-DMSO): δ=0.75 (d, 3H), 0.78 (d, 6H), 0.80 (t, 3H), 0.82 (d, 3H), 0.90 (d, 3H), 0.91 (d, 3H), 0.92 (d, 3H), 0.95 (d, 3H), 0.96 (d, 3H), 1.05 (m, 1H), 1.19 (d, 3H), 1.25 (m, 2H), 1.50 (m, 4H), 1.51 (m, 2H), 1.55 (m, 1H), 1.61 (m, 1H), 1.65 (m, 1H), 1.84 (m, 1H), 1.85 (m, 1H), 1.86 (m, 1H), 1.89 (m, 1H), 1.95 (m, 1H), 2.75 (m, 2H), 3.40 (m, 1H), 3.52 (m, 2H), 3.53 (dd, 1H), 3.64 (m, 2H), 3.66 (m, 1H), 3.68 (dd, 1H), 3.73 (m, 2H), 4.00 (dd, 1H), 4.02 (br., 1H), 4.13 (br., 1H), 4.32 (dd, 1H), 4.39 (t, 1H), 4.55 (m, 1H), 4.75 (dd, 1H), 5.19 (t, 1H), 5.29 (d, 1H), 5.30 (br., 1H), 5.58 (m, 2H), 6.68 (m, 3H), 6.89 (d, 1H), 6.93 (m, 3H), 6.94 (br., 1H), 6.98 (d, 1H), 7.12 (br., 1H), 7.20 (br., 2H), 7.23 (m, 2H), 7.42 (m, 2H), 7.54 (d, 1H), 7.58 (d, 1H), 8.32 (br., 1H), 9.18 (br., 1H), 9.20 (m, 2H), 9.50 (br., 1H).

¹³C-NMR (125.77 MHz, d₆-DMSO): δ=10.3, 15.3, 19.0, 19.2, 19.6, 20.0, 20.9, 22.0, 22.4, 23.0, 23.2, 24.3, 24.4, 25.0, 25.4, 26.0, 27.8, 30.9, 35.4, 39.5, 40.8, 40.9, 41.6, 44.1, 51.5, 52.7, 55.9, 56.2, 56.4, 57.9, 58.8, 60.2, 61.1, 62.6, 70.1, 71.6, 71.7, 75.5, 128.1, 128.6, 136.7, 156.8, 168.2, 170.1, 170.4, 171.2, 171.5, 171.9, 172.2, 172.4, 173.7.

The assignment of the signals took place in accordance with the assignment described in the literature (T. Kato, H. Hinoo, Y. Terui, J. Antibiot., 1988, 61, 719-725).

### Example 2A

D-Leucyl-L-leucyl-[(3R)-3-hydroxy-L-phenylalanyl]-[(3R)-3-hydroxy-L-leucyl]-L-leucyl-D-arginyl-L-isoleucyl-L-allothreonylglycyl-[(3S)-3-hydroxy-L-aspartyl]-L-serine C^{1.11}—O^{3.3}-lactone bistrifluoroacetate, [10-{(3S)-3-hydroxy-L-aspartate}]lysobactin Bistrifluoro-Acetate

(Lysobactin Acid Bistrifluoroacetate)

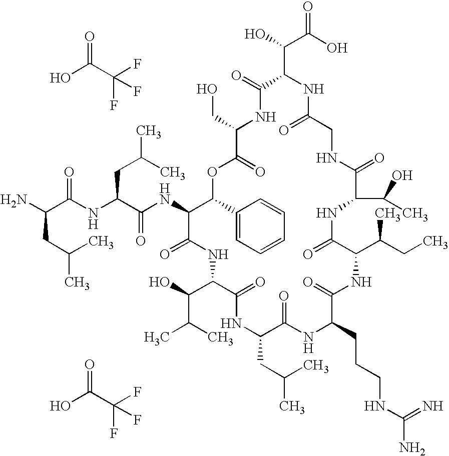

A suspension of lysobactin bistrifluoroacetate (example 1A, 30.0 mg, 19.94 μmol) in dioxane/10% water (1.5 ml) is mixed with 6 N hydrochloric acid (6 ml) and stirred at RT until conversion is complete after 2 days. The reaction is continually checked by HPLC. For the workup, the solvent is removed on a rotary evaporator at a bath temperature of 30° C., and the residue is purified by preparative RP-HPLC (Method 6) at RT. 21 mg (76% of theory) of product are obtained.

HPLC (Method 16) Rₜ=37.46 min.

LC-MS (Method 10): Rₜ=1.60 min; MS (ESIpos): m/z (%)=1277 (5) [M+H]⁺, 639 (100) [M+2H]²⁺; MS (ESIneg): m/z (%)=1275 (60) [M−H]⁻, 637 (100) [M−2H]²⁻.

HR-TOF-MS (Method 11): C₅₈H₉₇N₁₄O₁₈ [M+H]⁺ found 1277.7104, calc. 1277.7100.

### Example 3A

N^{2.1}-(Benzyloxycarbonyl)-D-leucyl-L-leucyl-[(3R)-3-hydroxy-L-phenylalanyl]-[(3R)-3-hydroxy-L-leucyl]-L-leucyl-D-arginyl-L-isoleucyl-L-allothreonylglycyl-[(3S)-3-hydroxy-L-aspartyl]-L-serine C^{1.11}—O^{3.3}-lactone Trifluoroacetate

(N^{2.1}-(Benzyloxycarbonyl)lysobactin Acid Trifluoroacetate)

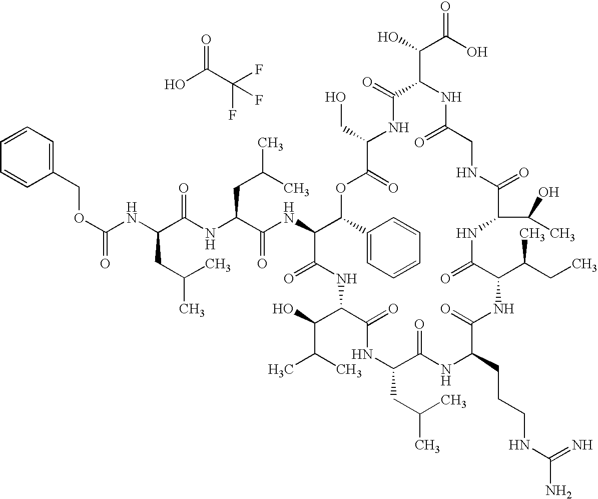

Lysobactin acid bistrifluoroacetate (example 2A, 200.0 mg, 0.13 μmol) is dissolved in a mixture of THF (30 ml) and DMF (5 ml) and then N-(benzyloxycarbonyloxy)succinimide (99.3 mg, 0.40 mmol) and NMM (39 μl, 36.3 mg, 0.36 mmol) are added at 0° C. The reaction is warmed to RT. The mixture is stirred overnight, during which complete conversion is observed. The solvent is removed on a rotary evaporator at a bath temperature of 30° C. and purified by preparative RP-HPLC (Method 8). Freeze drying results in 99.0 mg (49% of theory) of the title compound.

HPLC (Method 15) Rₜ=18.75 min

LC-MS (Method 10): Rₜ=2.27 min; MS (ESIpos): m/z (%)=1411 (37) [M+H]⁺, 706 (100) [M+2H]²⁺; MS (ESIneg): m/z (%)=1410 (100) [M−H]⁻, 704 (40) [M−2H]²⁻.

HR-TOF-MS (Method 11): C₆₆H₁₀₃N₁₄O₂₀ [M+H]⁺ found 1411.7493, calc. 1411.7468.

### Example 4A

N^{2.1}-(tert-Butoxycarbonyl)-D-leucyl-L-leucyl-[(3R)-3-hydroxy-L-phenylalanyl]-[(3R)-3-hydroxy-L-leucyl]-L-leucyl-D-arginyl-L-isoleucyl-L-allothreonylglycyl-[(3S)-3-hydroxy-L-aspartyl]-L-serine C^{1.11}—O^{3.3}-lactone Trifluoroacetate

(N^{2.2}-(tert-Butoxycarbonyl)lysobactin Acid Trifluoroacetate)

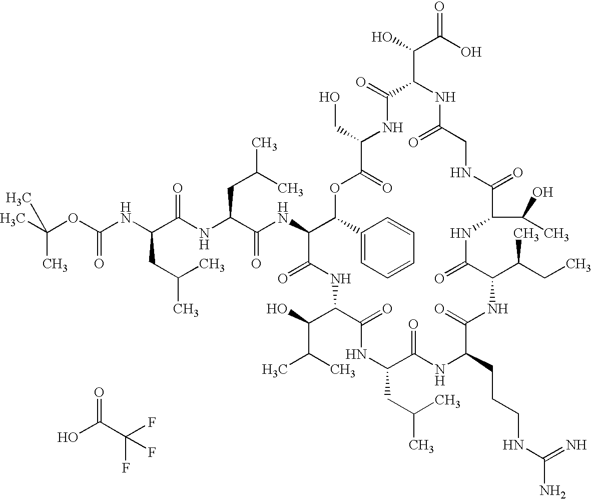

Under an argon protective gas atmosphere, lysobactin acid bistrifluoroacetate (example 2A, 200.0 mg, 0.13 mmol) is dissolved in a mixture of dioxane (22.4 ml), buffer of pH 6 (11.2 ml, Riedel de Haën, with fungicide) and phosphate buffer of pH 7 (11.2 ml, Dr. Lang, LCX021). Then, at 0° C., di-tert-butyl dicarbonate (34.8 mg, 0.16 mmol, 1.2 equivalents) and N,N-diisopropylethylamine (28 μl, 20.6 mg, 0.16 mmol, 1.2 equivalents) are successively added, and the mixture is stirred at RT overnight. After further addition of di-tert-butyl dicarbonate (29.0 mg, 0.13 mmol, 1 equivalent) and N,N-diisopropylethylamine (14 μl, 10.3 mg, 0.08 mmol, 0.6 equivalents) at RT and stirring at RT for 3 hours, complete conversion is achieved. For the workup, the reaction solution is directly added onto the preparative RP-HPLC (Method 7). 147.9 mg (75% of theory) of the title compound are obtained.

HPLC (Method 15) Rₜ=23.10 min

LC-MS (Method 10): Rₜ=2.28 min; MS (ESIpos): m/z (%)=1378 (34) [M+H]⁺, 639 (100); MS (ESIneg): m/z (%)=1376 (73) [M−H]⁻, 688 (100) [M−2H]²⁻.

### Example 5A

N^{2.1}-[Benzyloxycarbonyl)]-N^{4.10}-(2-morpholin-4-ylethyl)-D-leucyl-L-leucyl-[(3R)-3-hydroxy-L-phenylalanyl]-[(3R)-3-hydroxy-L-leucyl]-L-leucyl-D-arginyl-L-isoleucyl-L-allothreonylglycyl-[(3S)-3-hydroxy-L-asparaginyl]-L-serine C^{1.11}—O^{3.3}-lactone Bistrifluoro-Acetate

(N^{2.1}-[Benzyloxycarbonyl]-N^{4.10}-(2-morpholin-4-ylethyl)lysobactin Bistrifluoroacetate)

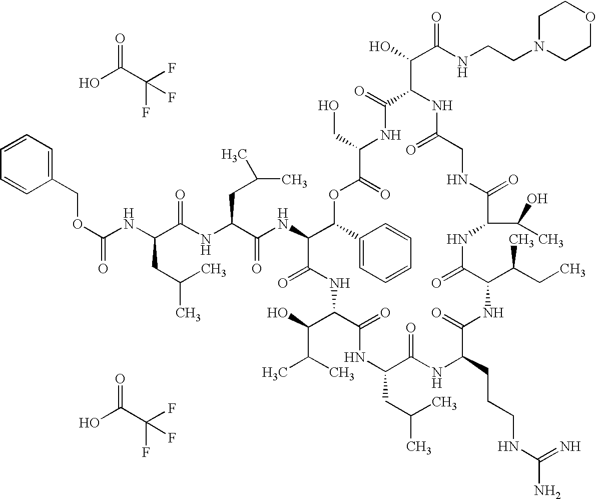

The cyclopeptide (example 3A, 4.0 mg, 2.62 μmol), N-(2-aminoethyl)morpholine (3 μl, 3.4 mg, 26.2 μmol, 10 equivalents) and HATU (11.7 mg, 31.46 μmol, 12 equivalents) are reacted in DMF (500 μl) according to procedure 3 at RT overnight to give the amide. Preparative HPLC (Method 6) results in 3.0 mg (65% of theory) of the title compound.

HPLC (Method 15): Rₜ=22.06 min.

LC-MS (Method 10): Rₜ=1.88 min; MS (ESIpos.): m/z (%)=763 (100) [M+2H]²⁺; ESIneg: m/z (%)=1522 (50) [M−H]⁻, 761 (100) [M−2H]²⁻.

HR-TOF-MS (Method 11): C₇₂H₁₁₅N₁₆O₂₀ calc. 1523.8469, found 1523.8517 [M+H]⁺.

### Example 6A

N^{2.1}-[Benzyloxycarbonyl]-N^{4.10}-(2-hydroxyethyl)-D-leucyl-L-leucyl-[(3R)-3-hydroxy-L-phenylalanyl]-[(3R)-3-hydroxy-L-leucyl]-L-leucyl-D-arginyl-L-isoleucyl-L-allothreonyl-glycyl-[(3S)-3-hydroxy-L-asparaginyl]-L-serine C^{1.11}—O^{3.3}-lactone Trifluoroacetate

(N^{2.1}-[Benzyloxycarbonyl]-N^{4.10}-(2-hydroxyethyl)lysobactin Trifluoroacetate)

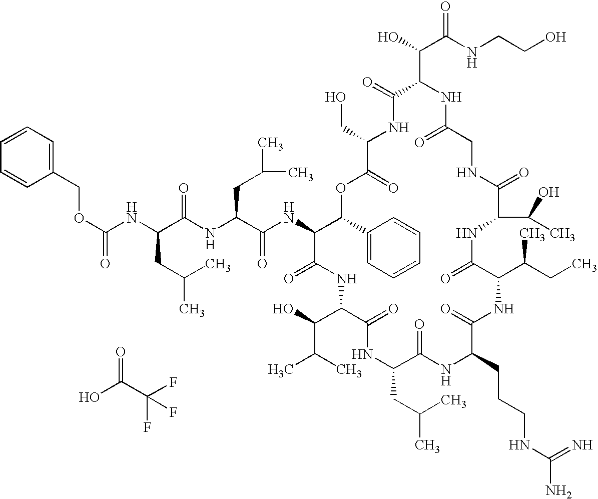

The cyclopeptide (example 3A, 20.0 mg, 13.11 μmol), ethanolamine (11 μl, 11.2 mg, 183.53 μmol, 14 equivalents), HATU (39.9 mg, 104.88 μmol, 8 equivalents) and NMM (12 μl, 10.8 mg, 104.88 μmol, 8 equivalents) are reacted in DMF (500 μl) according to procedure 3 at RT overnight to give the amide. Preparative HPLC (Method 6) results in 15.7 mg (76% of theory) of the title compound.

HPLC (Method 15): Rₜ=21.82 min.

LC-MS (Method 10): Rₜ=2.07 min; MS (ESIpos.): m/z (%)=1455 (35) [M+H]⁺; 728 (100) [M+2H]²⁺, ESIneg: m/z (%)=1523 (60) [M−H]⁻, 726 (12) [M−2H]²⁻, 672 (100).

HR-TOF-MS (Method 11): C₆₈H₁₀₈N₁₅O₂₀ calc. 1454.7890, found 1454.7860 [M+H]⁺.

### Example 7A

N^{2.1}-[Benzyloxycarbonyl]-N^{4.10}-methyl-D-leucyl-L-leucyl-[(3R)-3-hydroxy-L-phenylalanyl]-[(3R)-3-hydroxy-L-leucyl]-L-leucyl-D-arginyl-L-isoleucyl-L-allothreonylglycyl-[(3S)-3-hydroxy-L-asparaginyl]-L-serine C^{1.11}—O^{3.3}-lactone Trifluoroacetate

(N^{2.1}-[Benzyloxycarbonyl]-N^{4.10}-methyllysobactin Trifluoroacetate)

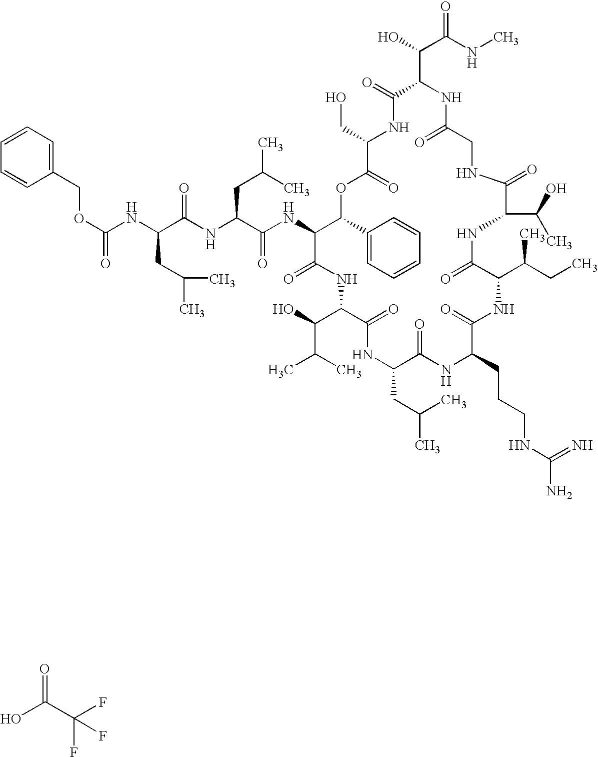

The cyclopeptide (example 3A, 20.0 mg, 13.11 μmol) and the methylamine hydrochloride (4.4 mg, 65.55 μmol, 5 equivalents) are reacted with the assistance of HATU (15.0 mg, 39.33 μmol, 3 equivalents) and NMM (20 μl, 18.56 mg, 183.54 μmol, 14 equivalents) in DMF (500 μl) according to procedure 3 at 4° C. within 3 days to give the amide. Preparative HPLC (Method 6) results in 17.0 mg (84% of theory) of the title compound.

HPLC (Method 15): Rₜ=21.26 min.

LC-MS (Method 10): Rₜ=2.07 min; MS (ESIpos.): m/z (%)=1425 (35) [M+H]⁺, 713 (100) [M+2H]²⁺; ESIneg: m/z (%)=1524 (100) [M−H]⁻, 711 (85) [M−2H]²⁻.

HR-TOF-MS (Method 11): C₆₇H₁₀₅N₁₅O₁₉ calc. 1424.7824, found 1424.7830 [M+H]⁺.

### Example 8A

N^{2.1}-[Benzyloxycarbonyl]-N^{4.10}-(3-morpholin-4-ylpropyl)-D-leucyl-L-leucyl-[(3R)-3-hydroxy-L-phenylalanyl]-[(3R)-3-hydroxy-L-leucyl]-L-leucyl-D-arginyl-L-isoleucyl-L-allothreonylglycyl-[(3S)-3-hydroxy-L-asparaginyl]-L-serine C^{1.11}—O^{3.3}-lactone Bistrifluoroacetate

(N^{2.1}-[Benzyloxycarbonyl]-N^{4.10}-(3-morpholin-4-ylpropyl)lysobactin Bistrifluoroacetate)

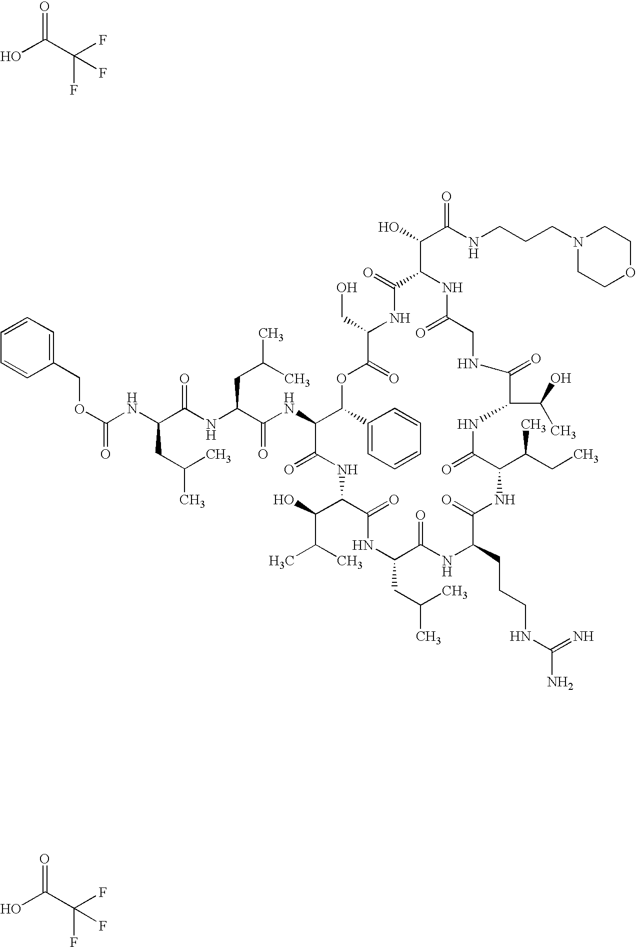

The cyclopeptide (example 3A, 13.0 mg, 8.52 μmol), 3-(morpholin-4-yl)propylamine (20 μl, 17.2 mg, 119.28 μmol, 14 equivalents), HATU (25.9 mg, 68.16 μmol, 8 equivalents) and NMM (7 μl, 6.9 mg, 68.16 μmol, 8 equivalents) are reacted in DMF (500 μl) according to procedure 3 at 4° C. within 3 days to give the amide. Preparative HPLC (Method 6) results in 12.0 mg (80% of theory) of the title compound.

HPLC (Method 15): Rₜ=22.15 min.

LC-MS (Method 10): Rₜ=2.07 min; MS (ESIpos.): m/z (%)=770 (100) [M+2H]²⁺; ESIneg: m/z (%)=1537 (100) [M−H]⁻, 768 (75) [M−2H]²⁻.

HR-TOF-MS (Method 11): C₇₃H₁₁₇N₁₆O₂₀ calc. 1537.8625, found 1537.8623 [M+H]⁺.

### Example 9A

N^{2.1}-[Benzyloxycarbonyl]-N^{4.10}-(pyridin-3-ylmethyl)-D-leucyl-L-leucyl-[(3R)-3-hydroxy-L-phenylalanyl]-[(3R)-3-hydroxy-L-leucyl]-L-leucyl-D-arginyl-L-isoleucyl-L-allothreonyl-glycyl-[(3S)-3-hydroxy-L-asparaginyl]-L-serine C^{1.11}—O^{3.3}-lactone Bistrifluoroacetate

(N^{2.1}-[Benzyloxycarbonyl]-N^{4.10}-(pyridin-3-ylmethyl)lysobactin Bistrifluoroacetate)

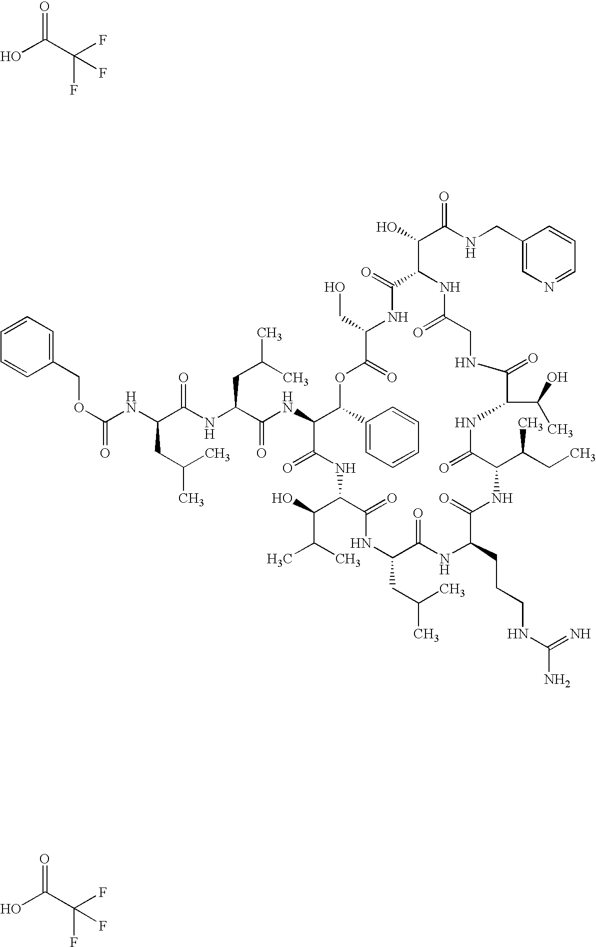

The cyclopeptide (example 3A, 20.0 mg, 13.11 μmol), 3-picolylamine (19.5 mg, 183.5 μmol, 14 equivalents), HATU (39.9 mg, 104.88 μmol, 8 equivalents) and NMM (12 μl, 10.6 mg, 104.88 μmol, 8 equivalents) are reacted in DMF (500 μl) according to procedure 3 at RT overnight to give the amide. Preparative HPLC (Method 6) results in 17.0 mg (84% of theory) of the title compound.

HPLC (Method 14): Rₜ=2.11 min.

LC-MS (Method 10): Rₜ=1.95 min; MS (ESIpos.): m/z (%)=1503 (5) [M+H]⁺, 751 (100) [M+2H]²⁺; ESIneg: m/z (%)=1500 (84) [M−H]⁻, 750 (30) [M−2H]²⁻, 695 (100).

HR-TOF-MS (Method 11): C₇₂H₁₀₇N₁₅O₂₀ calc. 1501.7812, found 1501.7819 [M+H]⁺.

### Example 10A

N^{2.1}-[Benzyloxycarbonyl]-N^{4.10}-benzyl-D-leucyl-L-leucyl-[(3R)-3-hydroxy-L-phenylalanyl]-[(3R)-3-hydroxy-L-leucyl]-L-leucyl-D-arginyl-L-isoleucyl-L-allothreonyl-glycyl-[(3S)-3-hydroxy-L-asparaginyl]-L-serine C^{1.11}—O^{3.3}-lactone Trifluoroacetate

(N^{2.1}-[Benzyloxycarbonyl]-N^{4.10}-benzyllysobactin Trifluoroacetate)

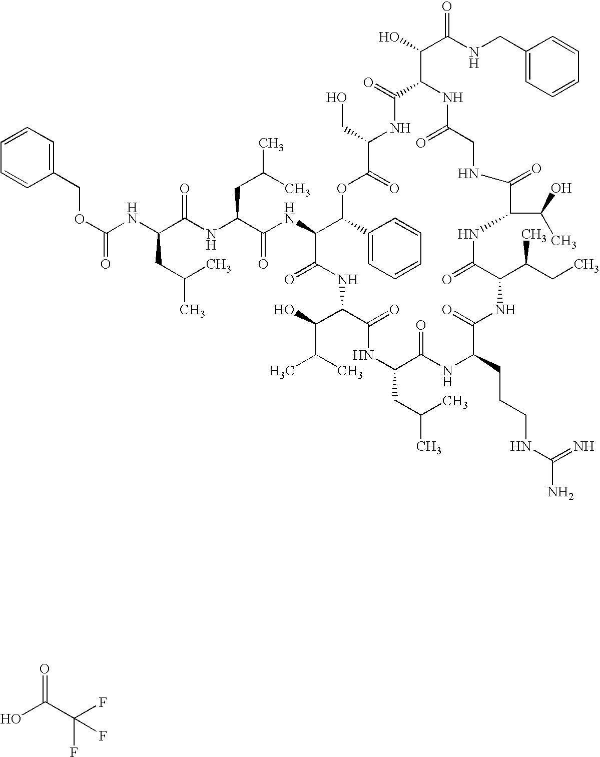

The cyclopeptide (example 3A, 20.0 mg, 13.11 μmol), benzylamine (20 μl, 19.7 mg, 183.54 μmol, 14 equivalents), HATU (39.9 mg, 104.88 μmol, 8 equivalents) and NMM (12 μl, 10.6 mg, 104.88 μmol, 8 equivalents) are reacted in DMF (500 μl) according to procedure 3 at RT overnight to give the amide. Preparative HPLC (Method 6) results in 17.6 mg (83% of theory) of the title compound.

HPLC (Method 15): Rₜ=23.21 min.

LC-MS (Method 10): Rₜ=2.14 min; MS (ESIpos.): m/z (%)=1502 (32) [M+H]⁺, 751 (100) [M+2H]²⁺; ESIneg: m/z (%)=1500 (75) [M−H]⁻, 749 (20) [M−2H]²⁻, 695 (100).

HR-TOF-MS (Method 11): C₇₃H₁₁₀N₁₅O₁₉ calc. 1500.8097, found 1500.8131 [M+H]⁺.

### Example 11A

N^{2.1}-(tert.-Butoxycarbonyl)-N^{4.10},N^{4.10}-dimethyl-D-leucyl-L-leucyl-[(3R)-3-hydroxy-L-phenylalanyl]-[(3R)-3-hydroxy-L-leucyl]-L-leucyl-D-arginyl-L-isoleucyl-L-allothreonyl-glycyl-[(3S)-3-hydroxy-L-asparaginyl]-L-serine C^{1.11}—O^{3.3}-lactone Trifluoroacetate

(N^{2.1}-(tert.-Butoxycarbonyl)-N^{4.10},N^{4.10}-dimethyllysobactin Trifluoroacetate)

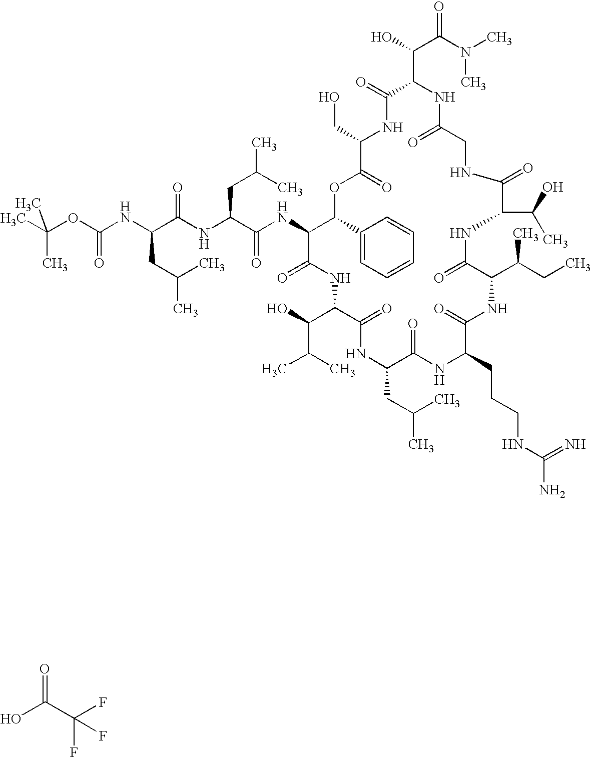

The cyclopeptide (example 4A, 20.0 mg, 13.41 μmol) and a 2M solution of dimethylamine (67 μl, 6.0 mg, 134.10 μmol, 10 equivalents) in THF are reacted with the assistance of HATU (25.5 mg, 67.05 μmol, 5 equivalents) and NMM (12 μl, 10.6 mg, 107.28 μmol, 8 equivalents) in DMF (500 μl) according to procedure 3 at 0° C. overnight to give the amide. Separation by preparative HPLC (Method 6) results in 8.5 mg (42% of theory) of the title compound.

HPLC (Method 15): Rₜ=23.89 min.

LC-MS (Method 10): Rₜ=2.26 min; MS (ESIpos.): m/z (%)=1405 (9) [M+H]⁺, 753 (100) [M+2H]²⁺; ESIneg: m/z (%)=1403 (100) [M−H]⁻.

HR-TOF-MS (Method 11): C₆₅H₁₁₀N₁₅O₁₉ calc. 1404.8097, found 1404.8094 [M+H]⁺.

### Example 12A

N^{2.1}-(tert-Butoxycarbonyl)-N^{4.10}-[2-(benzyloxy)-2-oxoethyl]-D-leucyl-L-leucyl-[(3R)-3-hydroxy-L-phenylalanyl]-[(3R)-3-hydroxy-L-leucyl]-L-leucyl-D-arginyl-L-isoleucyl-L-allothreonylglycyl-[(3S)-3-hydroxy-L-β-asparaginyl]-L-serine C^{1.11}—O^{3.3}-lactone Trifluoroacetate

(N^{2.1}-(tert-Butoxycarbonyl)-N^{4.10}-[2-(benzyloxy)-2-oxoethyl]lysobactin Trifluoroacetate)

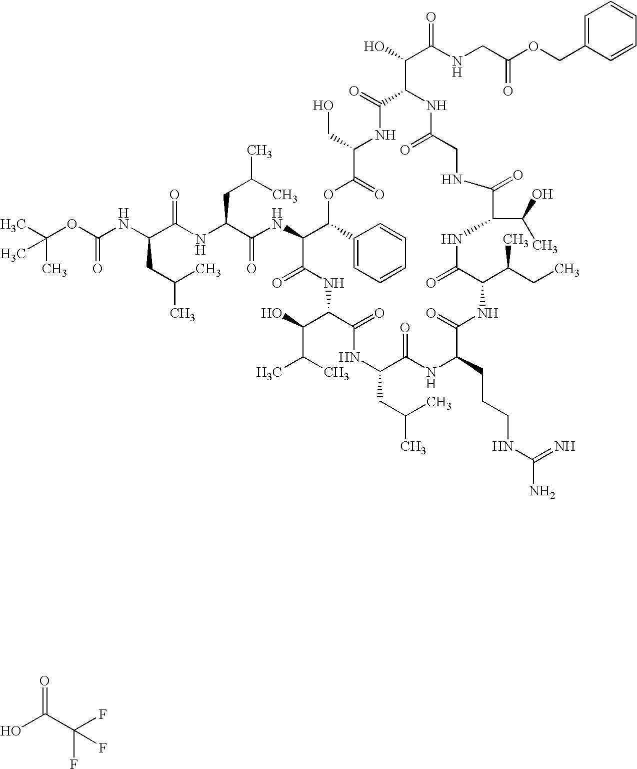

The cyclopeptide (example 4A, 20.0 mg, 13.41 μmol), glycine benzyl ester hydrochloride (21.6 mg, 107.28 μmol, 8 equivalents), HATU (25.5 mg, 67.05 μmol, 5 equivalents) and NMM (29 μl, 27.1 mg, 268.2 μmol, 20 equivalents) are reacted in DMF (500 μl) according to procedure 3 at RT overnight to give the amide. Purification by chromatography (Method 6) results in 21.5 mg (98% of theory) of the title compound.

HPLC (Method 15): Rₜ=23.60 min.

LC-MS (Method 10): Rₜ=2.32 min; MS (ESIpos.): m/z (%)=1525 (40) [M+H]⁺, 763 (38) [M+2H]²⁺, 713 (100); ESIneg: m/z=1523 (60) [M−H]⁻, 761 (100) [M−2H]²⁻.

HR-TOF-MS (Method 11): C₇₂H₁₁₄N₁₅O₂₁ calc. 1524.8309, found 1524.8311 [M+H]⁺.

### Example 13A

N^{2.1}-(tert-Butoxycarbonyl)-N^{4.10}-(2-{[benzyloxycarbonyl]amino}ethyl)-D-leucyl-L-leucyl-[(3R)-3-hydroxy-L-phenylalanyl]-[(3R)-3-hydroxy-L-leucyl]-L-leucyl-D-arginyl-L-iso-leucyl-L-allothreonylglycyl-[(3S)-3-hydroxy-L-asparaginyl]-L-serine C^{1.11}-O^{3.3}-lactone Trifluoroacetate

(N^{2.1}-(tert-Butoxycarbonyl)-N^{4.10}-(2-{[benzyloxycarbonyl]amino}ethyl)lysobactin Trifluoroacetate)

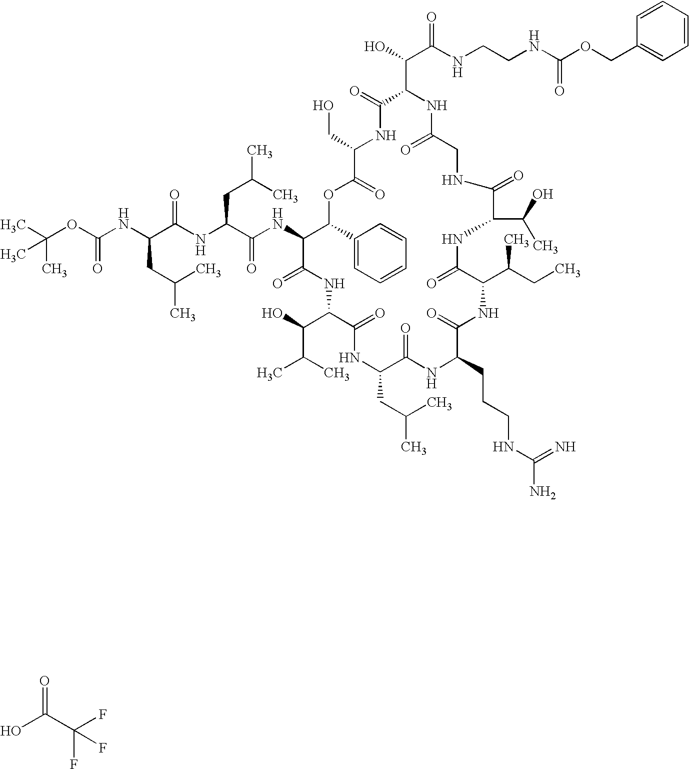

The cyclopeptide (example 4A, 40.0 mg, 26.82 μmol), benzyl (2-aminoethyl)carbamate hydrochloride (49.5 mg, 214.53 μmol, 8 equivalents) are reacted according to procedure 3 with the assistance of HATU (50.982 mg, 134.08 μmol, 5 equivalents) and NMM (59 μl, 54.2 mg, 536.31 μmol, 20 equivalents) in DMF (500 μl) at RT overnight to give the amide. Purification by chromatography (Method 6) results in 40.2 mg (90% of theory) of the title compound.

HPLC (Method 15): Rₜ=23.64 min.

LC-MS (Method 10): Rₜ=2.30 min; MS (ESIpos.): m/z (%)=1554 (100) [M+H]⁺, 777 (53) [M+2H]²⁺; ESIneg: m/z (%)=1552 (19) [M−H]⁻, 775 (100) [M−2H]²⁻.

HR-TOF-MS (Method 11): C₇₃H₁₁₇N₁₆O₂₁ calc. 1553.8574, found 1553.8595 [M+H]⁺.

### Example 14A

N^{4.10}-(2-Amino-2-oxoethyl)-N^{2.1}-(tert-butoxycarbonyl)-D-leucyl-L-leucyl-[(3R)-3-hydroxy-L-phenylalanyl]-[(3R)-3-hydroxy-L-leucyl]-L-leucyl-D-arginyl-L-isoleucyl-L-allothreonylglycyl-[(3S)-3-hydroxy-L-asparaginyl]-L-serine C^{1.11}—O^{3.3}-lactone Trifluoroacetate

(N^{4.10}-(2-Amino-2-oxoethyl)-N^{2.1}-(tert-butoxycarbonyl)lysobactin Trifluoroacetate)

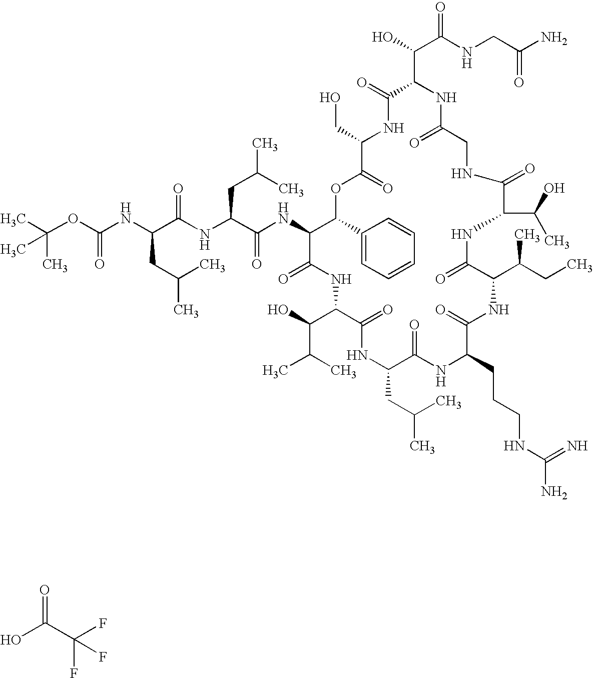

The cyclopeptide (example 4A, 20.0 mg, 13.41 μmol) and glycinamine hydrochloride (11.9 mg, 107.28 μmol, 8 equivalents) are reacted according to procedure 3 with the assistance of HATU (25.5 mg, 67.05 μmol, 5 equivalents) and NMM (29 μl, 10.6 mg, 268.20 μmol, 20 equivalents) in DMF (500 μl) at RT overnight to give the amide. Purification by chromatography (Method 6) results in 12.8 mg (60% of theory) of the title compound.

HPLC (Method 15): Rₜ=21.97 min.

LC-MS (Method 10): Rₜ=2.19 min; MS (ESIpos.): m/z (%)=1435 (23) [M+H]⁺, 718 (18) [M+2H]²⁺, 667 (100); ESIneg: m/z (%)=1433 (13) [M−H]⁻, 716 (100) [M−2H]²⁻.

HR-TOF-MS (Method 11): C₆₅H₁₀₉N₁₆O₂₀ calc. 1433.7999, found 1433.8000 [M+H]⁺.

### Example 15A

N^{2.1}-(tert-Butoxycarbonyl)-N^{4.10}-[2-(2-{[bis(dimethylamino)methylene]amino}ethoxy)ethyl]-D-leucyl-L-leucyl-[(3R)-3-hydroxy-L-phenylalanyl]-[(3R)-3-hydroxy-L-leucyl]-L-leucyl-D-arginyl-L-isoleucyl-L-allothreonylglycyl-[(3S)-3-hydroxy-L-asparaginyl]-L-serine C^{1.11}—O^{3.3}-lactone Bistrifluoroacetate

(N^{2.1}-(tert-Butoxycarbonyl)-N^{4.10}-[2-(2-{[bis(dimethylamino)methylene]amino}ethoxy)ethyl]lysobactin Bistrifluoroacetate)

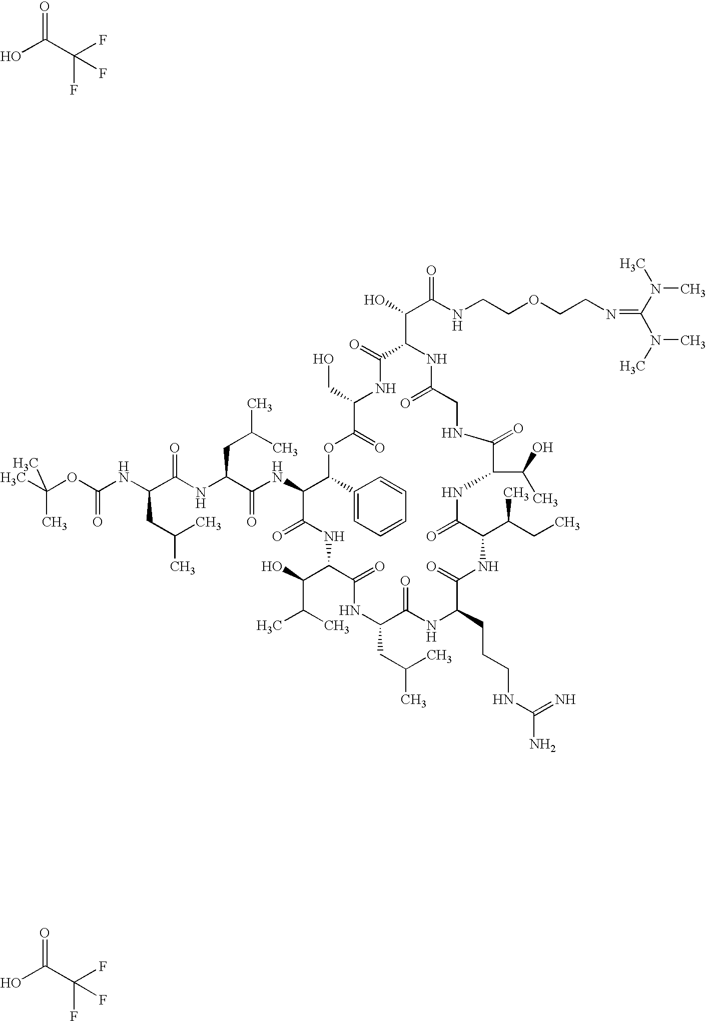

The cyclopeptide (example 4A, 20.0 mg, 13.41 μmol) and 2,2′-oxydiethylamine hydrochloride (1.4 mg, 8.05 μmol, 0.6 equivalents) are reacted according to procedure 3 with the assistance of HATU (15.3 mg, 40.23 μmol, 3 equivalents) and NMM (12 μl, 10.9 mg, 107.28 μmol, 8 equivalents) in DMF (500 μl) at RT overnight to give the amide. Purification by chromatography (Method 6) results in 5.3 mg (22% of theory) of the title compound.

HPLC (Method 15): Rₜ=21.81 min.

LC-MS (Method 10): Rₜ=2.02 min; MS (ESIpos.): m/z (%)=782 (100) [M+2H]²⁺; ESIneg: m/z (%)=1560 (83) [M−H]⁻, 780 (28) [M−2H]²⁻, 1606 (100).

HR-TOF-MS (Method 11): C₇₂H₁₂₅N₁₈O₂₀ calc. 1561.9313, found 1561.9352 [M+H]⁺.

### Example 16A

N^{4.10}-(2-Aminoethyl)-N^{2.1}-(tert-butoxycarbonyl)-D-leucyl-L-leucyl-[(3R)-3-hydroxy-L-phenylalanyl]-[(3R)-3-hydroxy-L-leucyl]-L-leucyl-D-arginyl-L-isoleucyl-L-allothreonyl-glycyl-[(3S)-3-hydroxy-L-asparaginyl]-L-serine C^{1.11}—O^{3.3}-lactone Bistrifluoroacetate

(N^{4.10}-(2-Aminoethyl)-N^{2.1}-(tert-butoxycarbonyl)lysobactin Bistrifluoroacetate)

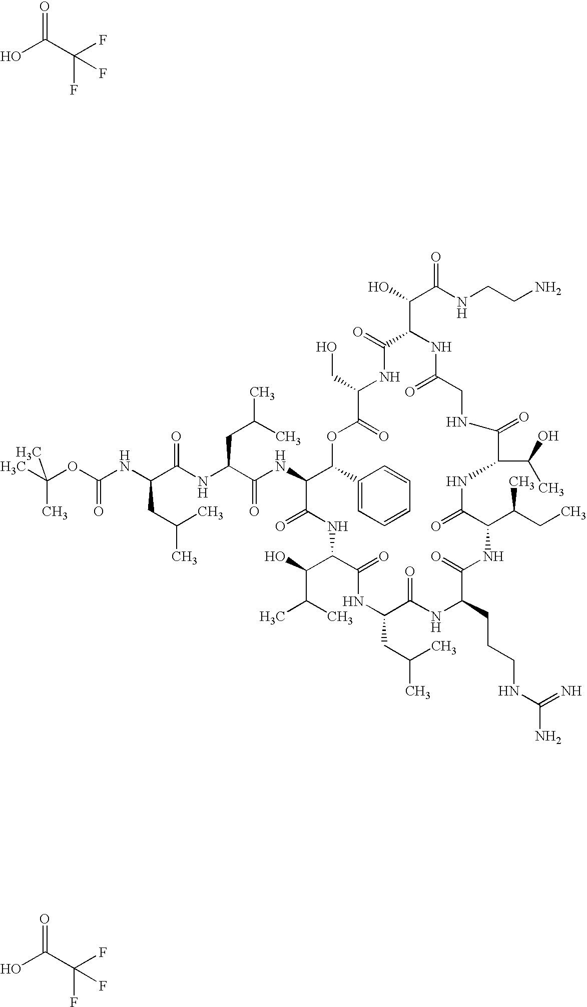

The Cbz protective group is removed from the compound of example 13A (19.0 mg, 11.39 μmol) by hydrogenolysis according to procedure 2. Fine purification by preparative HPLC (Method 6) results in 14.8 mg (78.9% of theory) of the title compound.

HPLC (Method 15): Rₜ=21.13 min.

LC-MS (Method 10): Rₜ=1.91 min; MS (ESIpos.): m/z (%)=1420 (5) [M+H]⁺, 710 (100) [M+2H]²⁺; ESIneg: m/z (%)=1418 (55) [M−H]⁻, 709 (100) [M−2H]²⁻.

HR-TOF-MS (Method 11): C₆₅H₁₁₁N₁₆O₁₉ calc. 1419.8206, found 1419.8221 [M+H]⁺.

### Example 17A

[3-tert-Butyl-D-alanyl]-[3-tert-butyl-L-alanyl]-[(3R)-3-hydroxy-L-phenylalanyl)]-[(3R)-3-hydroxy-L-leucyl]-L-leucyl-D-arginyl-L-isoleucyl-L-allothreonylglycyl-[(3S)-3-hydroxy-L-aspartyl]-L-serine C^{1.11}-O^{3.3}-lactone Bistrifluoroacetate

(C^{4.1},C^{4.2}-Dimethyllysobactin Acid Bistrifluoroacetate)

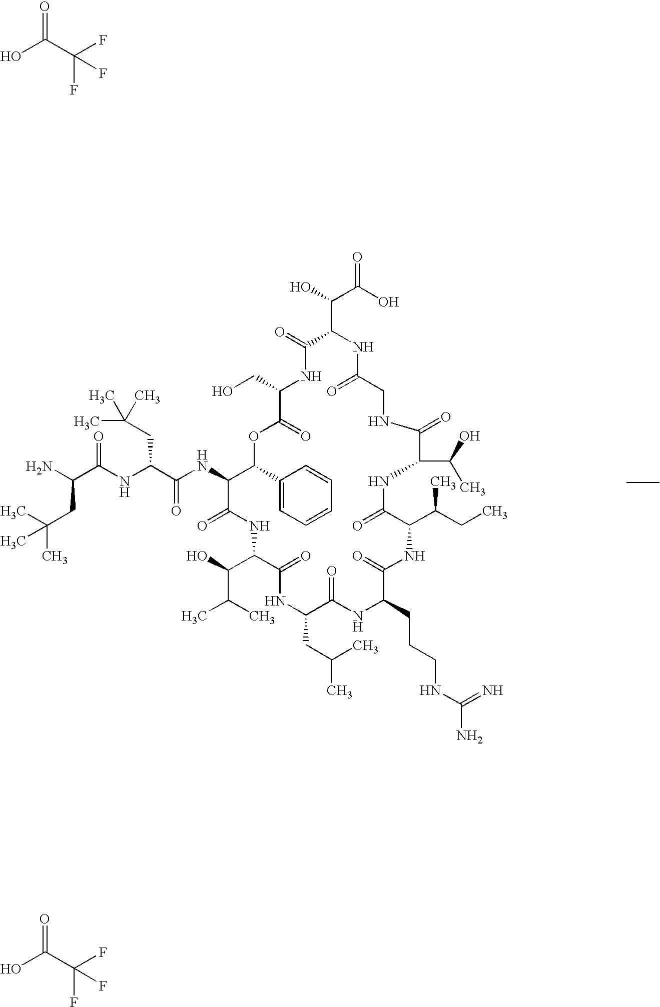

A suspension of [3-tert-butyl-D-alanyl]-[3-tert-butyl-L-alanyl]-[(3R)-3-hydroxy-L-phenylalanyl)]-[(3R)-3-hydroxy-L-leucyl]-L-leucyl-D-arginyl-L-isoleucyl-L-allothreonylglycyl-[(3S)-3-hydroxy-L-asparaginyl]-L-serine C^{1.11}—O^{3.3}-lactone bistrifluoroacetate (150.0 mg, 98 μmol) in dioxane/50% water (3 ml) is mixed with 6 N hydrochloric acid (9 ml) and stirred until conversion is complete in 5 days. The reaction is continually checked by HPLC. For the workup, the solvent is removed on a rotary evaporator at a bath temperature of 30° C., and the residue is purified by preparative RP-HPLC (Method 6). 92.8 mg (67% of theory) of the title compound are obtained.

HPLC (Method 3) Rₜ=1.85 min

LC-MS (Method 10): Rₜ=1.74 min, MS (ESIpos.): m/z (%)=653.7 (100) [M+2H]²⁺, 1306.0 (10) [M+H]⁺; MS (ESIneg): m/z (%)=651.7 (100) [M−H]⁻, 1339.9 (90) [M−H]⁻.

HR-TOF-MS (Method 11): C₆₀H₁₀₁N₁₄O₁₈ [M+H]⁺ calc.: 1305.7413, found: 1305.7433.

### Exemplary Embodiments

### Example 1

N^{4.10}-(3-Morpholin-4-ylpropyl)-D-leucyl-L-leucyl-[(3R)-3-hydroxy-L-phenylalanyl]-[(3R)-3-hydroxy-L-leucyl]-L-leucyl-D-arginyl-L-isoleucyl-L-allothreonylglycyl-[(3S)-3-hydroxy-L-asparaginyl]-L-serine C^{1.11}—O^{3.3}-lactone Tristrifluoroacetate

(N^{4.10}-(3-Morpholin-4-ylpropyl)lysobactin Tristrifluoroacetate)

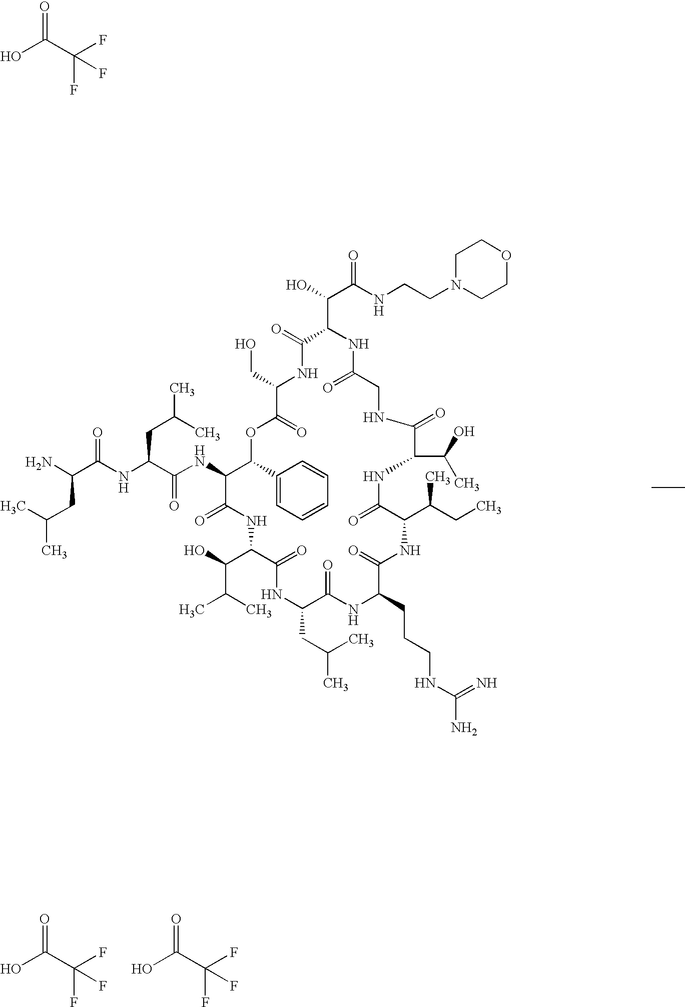

The benzyloxycarbonyl protecting group is removed from the compound of example 5A (2.5 mg, 1.43 μmol) by hydrogenolysis according to procedure 2. Fine purification (Method 6) and freeze drying result in the title compound (2.0 mg, 81% of theory) as an amorphous solid.

HPLC (Method 14): Rₜ=1.57 min.

LC-MS (Method 10): Rₜ=1.91 min; MS (ESIpos.): m/z (%)=1390 (3) [M+H]⁺, 695 (94) [M+2H]²⁺, 464 (100); ESIneg: m/z (%)=1388 (88) [M−H]⁻, 693 (100) [M−2H]²⁻.

HR-TOF-MS (Method 11): C₆₄H₁₀₉N₁₆O₁₈ calc. 1389.8101, found 1389.8116 [M+H]⁺.

### Example 2

N^{4.10}-(2-Hydroxyethyl)-D-leucyl-L-leucyl-[(3R)-3-hydroxy-L-phenylalanyl]-[(3R)-3-hydroxy-L-leucyl]-L-leucyl-D-arginyl-L-isoleucyl-L-allothreonylglycyl-[(3S)-3-hydroxy-L-asparaginyl]-L-serine C^{1.11}—O^{3.3}-lactone Bistrifluoroacetate

(N^{4.10}-(2-Hydroxyethyl)lysobactin Bistrifluoroacetate)

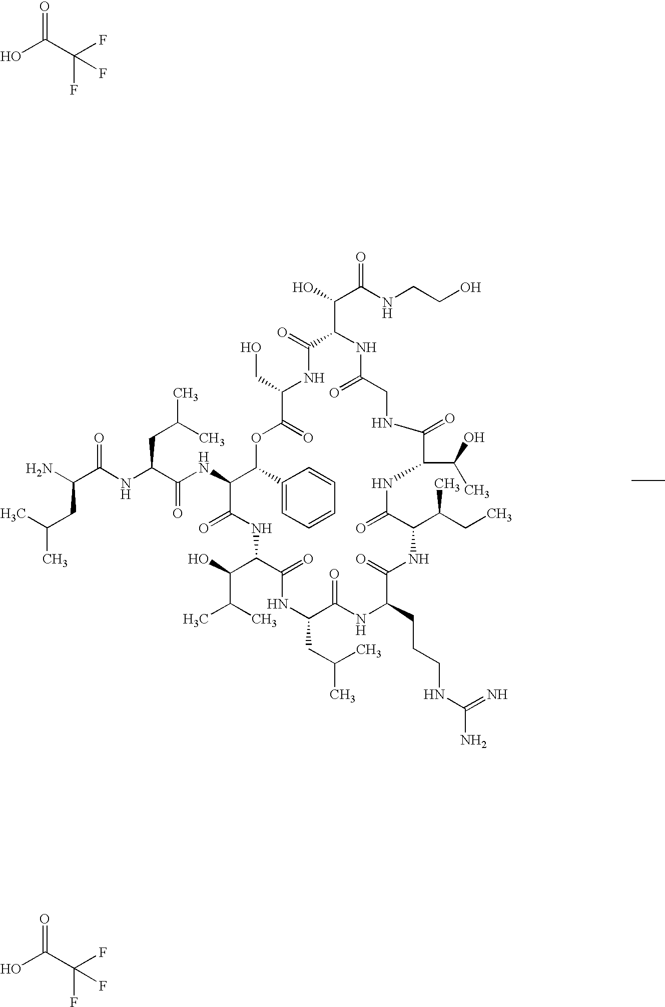

The benzyloxycarbonyl protecting group is removed from the compound of example 6A (14.5 mg, 9.24 μmol) by hydrogenolysis according to procedure 2. Fine purification (Method 6) and freeze drying result in the title compound (9.4 mg, 66% of theory) as an amorphous solid.

HPLC (Method 15): Rₜ=15.70 min.

LC-MS (Method 10): Rₜ=1.50 min; MS (ESIpos.): m/z (%)=1321 (8) [M+H]⁺, 661 (100) [M+2H]²⁺; ESIneg: m/z (%)=1318 (72) [M−H]⁻, 659 (100) [M−2H]²⁻.

HR-TOF-MS (Method 11): C₆₀H₁₀₂N₁₅O₁₈ calc. 1320.7522, found 1320.7504 [M+H]⁺.

### Example 3

N^{4.10}-Methyl-D-leucyl-L-leucyl-[(3R)-3-hydroxy-L-phenylalanyl]-[(3R)-3-hydroxy-L-leucyl]-L-leucyl-D-arginyl-L-isoleucyl-L-allothreonylglycyl-[(3S)-3-hydroxy-L-asparaginyl]-L-serine C^{1.11}—O^{3.3}-lactone Bistrifluoroacetate

(N^{4.10}-Methyllysobactin Bistrifluoroacetate)

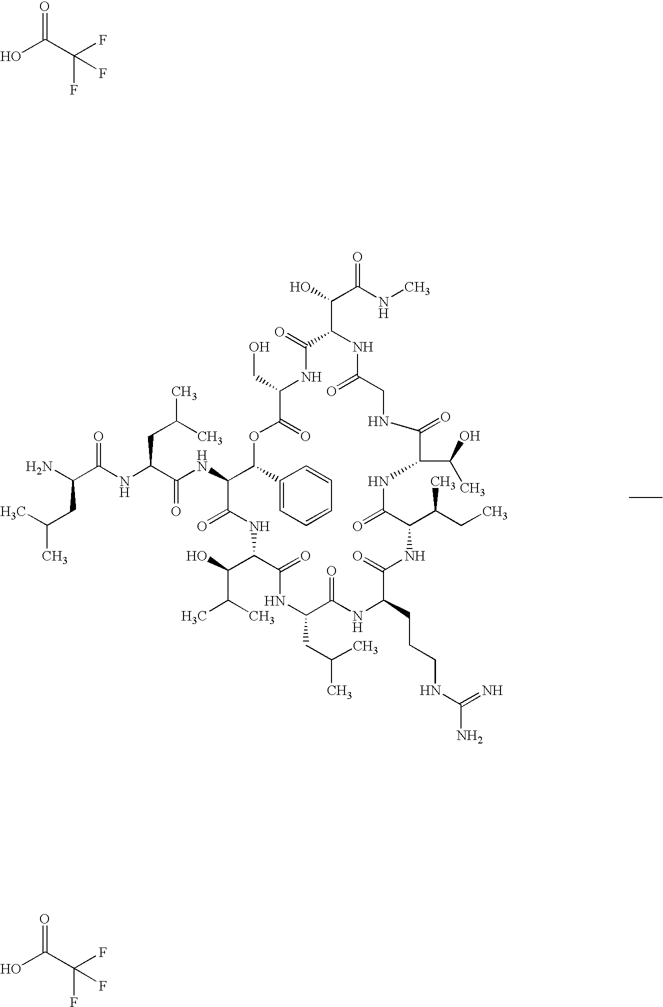

The benzyloxycarbonyl protecting group is removed from the compound of example 7A (16.0 mg, 10.40 μmol) by hydrogenolysis according to procedure 2. Fine purification (Method 6) and freeze drying result in the title compound (10.9 mg, 69% of theory) as an amorphous solid.

HPLC (Method 15): Rₜ=15.79 min.

LC-MS (Method 10): Rₜ=1.50 min; MS (ESIpos.): m/z (%)=1291 (12) [M+H]⁺, 646 (100) [M+2H]²⁺; ESIneg: m/z (%)=1289 (100) [M−H]⁻, 644 (37) [M−2H]²⁻.

HR-TOF-MS (Method 11): C₅₉H₁₀₀N₁₅O₁₇ calc. 1290.7417, found 1290.7404 [M+H]⁺.

### Example 4

N^{4.10}-(3-Morpholin-4-yl-propyl)-D-leucyl-L-leucyl-[(3R)-3-hydroxy-L-phenylalanyl]-[(3R)-3-hydroxy-L-leucyl]-L-leucyl-D-arginyl-L-isoleucyl-L-allothreonylglycyl-[(3S)-3-hydroxy-L-asparaginyl]-L-serine C^{1.11}—O^{3.3}-lactone Tristrifluoroacetate

(N^{4.10}-(3-Morpholin-4-yl-propyl)lysobactin Tristrifluoroacetate)

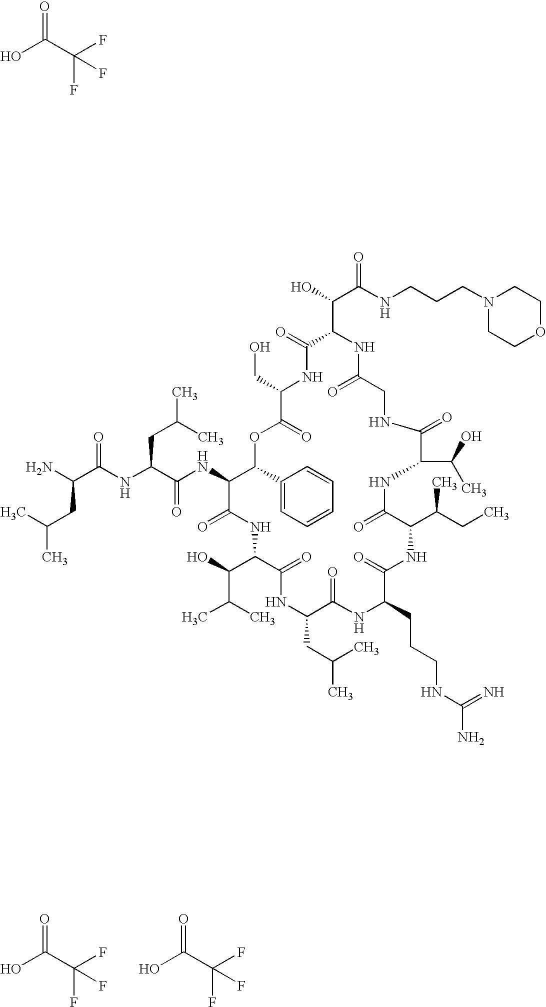

The benzyloxycarbonyl protecting group is removed from the compound of example 8A (11.0 mg, 6.23 μmol) by hydrogenolysis according to procedure 2. Fine purification (Method 6) and freeze drying result in the title compound (9.2 mg, 85% of theory) as an amorphous solid.

HPLC (Method 15): Rₜ=15.61 min.

LC-MS (Method 10): Rₜ=1.38 min; MS (ESIpos.): m/z (%)=702 (58) [M+2H]²⁺, 469 (100); ESIneg: m/z (%)=1402 (22) [M−H]⁻, 701 (100) [M−2H]²⁻.

HR-TOF-MS (Method 11): C₆₅H₁₁₁N₁₆O₁₈ calc. 1403.8257, found 1403.8289 [M+H]⁺.

### Example 5

N^{4.10}-(Pyridin-3-yl-methyl)-D-leucyl-L-leucyl-[(3R)-3-hydroxy-L-phenylalanyl]-[(3R)-3-hydroxy-L-leucyl]-L-leucyl-D-arginyl-L-isoleucyl-L-allothreonylglycyl-[(3S)-3-hydroxy-L-asparaginyl]-L-serine C^{1.11}—O^{3.3}-lactone Tristrifluoroacetate

(N^{4.10}-(Pyridin-3-yl-methyl)lysobactin Tristrifluoroacetate)

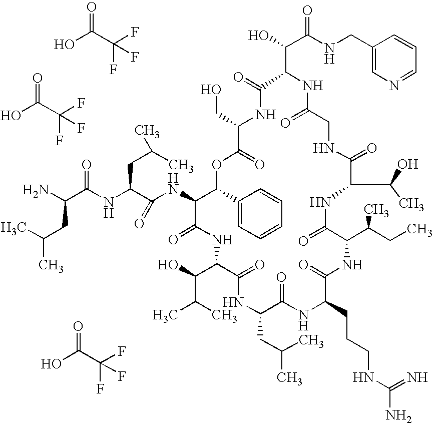

The benzyloxycarbonyl protecting group is removed from the compound of example 9A (8.5 mg, 4.91 μmol) by hydrogenolysis according to procedure 2. Fine purification (Method 6) and freeze drying result in the title compound (7.5 mg, 89% of theory) as an amorphous solid.

HPLC (Method 15): Rₜ=15.56 min.

LC-MS (Method 10): Rₜ=1.45 min; MS (ESIpos.): m/z (%)=1368 (5) [M+H]⁺, 684 (72) [M+2H]²⁺, 456 (100); ESIneg: m/z (%)=1366 (72) [M−H]⁻, 682 (100) [M−2H]²⁻.

HR-TOF-MS (Method 11): C₆₄H₁₀₃N₁₆O₁₇ calc. 1367.7682, found 1367.7684 [M+H]⁺.

### Example 6

N^{4.10}-Benzyl-D-leucyl-L-leucyl-[(3R)-3-hydroxy-L-phenylalanyl]-[(3R)-3-hydroxy-L-leucyl]-L-leucyl-D-arginyl-L-isoleucyl-L-allothreonylglycyl-[(3S)-3-hydroxy-L-asparaginyl]-L-serine C^{1.11}—O^{3.3}-lactone Bistrifluoroacetate

(N^{4.10}-Benzyllysobactin Bistrifluoroacetate)

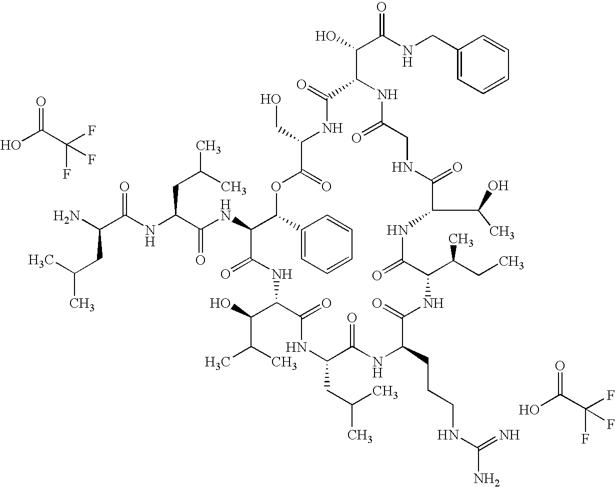

The benzyloxycarbonyl protecting group is removed from the compound of example 10A (16.0 mg, 9.91 μmol) by hydrogenolysis according to procedure 2. Fine purification (Method 6) and freeze drying result in the title compound (12.7 mg, 80% of theory) as an amorphous solid.

HPLC (Method 15): Rₜ=17.09 min.

LC-MS (Method 10): Rₜ=1.60 min; MS (ESIpos.): m/z (%)=1366 (15) [M+H]⁺, 684 (100) [M+2H]²⁺; ESIneg: m/z (%)=1364 (100) [M−H]⁻, 682 (100) [M−2H]²⁻.

HR-TOF-MS (Method 11): C₆₅H₁₀₄N₁₅O₁₇ calc. 1366.7730, found 1366.7698 [M+H]⁺.

### Example 7

N^{4.10},N^{4.10}-Dimethyl-D-leucyl-L-leucyl-[(3R)-3-hydroxy-L-phenylalanyl]-[(3R)-3-hydroxy-L-leucyl]-L-leucyl-D-arginyl-L-isoleucyl-L-allothreonylglycyl-[(3S)-3-hydroxy-L-asparaginyl]-L-serine C^{1.11}—O^{3.3}-lactone Bistrifluoroacetate

(N^{4.10},N^{4.10}-Dimethyllysobactin Bistrifluoroacetate)

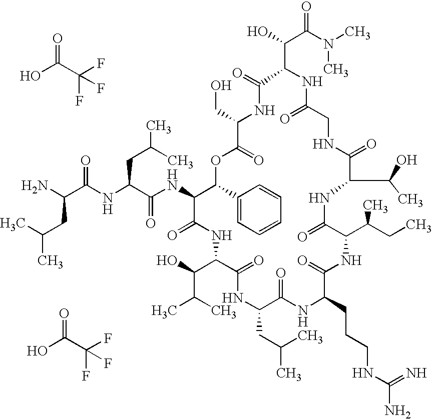

The Boc protecting group is removed from the compound of example 11A (7.0 mg, 4.61 μmol) according to procedure 1. Fine purification (Method 6) and freeze drying result in the title compound (3.6 mg, 51% of theory) as an amorphous solid.

HPLC (Method 15): Rₜ=16.13 min.

LC-MS (Method 10): Rₜ=1.50 min; MS (ESIpos.): m/z (%)=1305 (15) [M+H]⁺, 653 (100) [M+2H]²⁺; ESIneg: m/z (%)=1303 (100) [M−H]⁻, 651 (100) [M−2H]²⁻.

HR-TOF-MS (Method 11): C₆₀H₁₀₂N₁₅O₁₇ calc. 1304.7573, found 1304.7615 [M+H]⁺.

### Example 8

N^{4.10}-(2-(Benzyloxy)-2-oxoethyl)-D-leucyl-L-leucyl-[(3R)-3-hydroxy-L-phenylalanyl]-[(3R)-3-hydroxy-L-leucyl]-L-leucyl-D-arginyl-L-isoleucyl-L-allothreonylglycyl-[(3S)-3-hydroxy-L-β-asparaginyl]-L-serine C^{1.11}—O^{3.3}-lactone Bistrifluoroacetate

(N^{4.10}-(2-(Benzyloxy)-2-oxoethyl)lysobactin Bistrifluoroacetate)

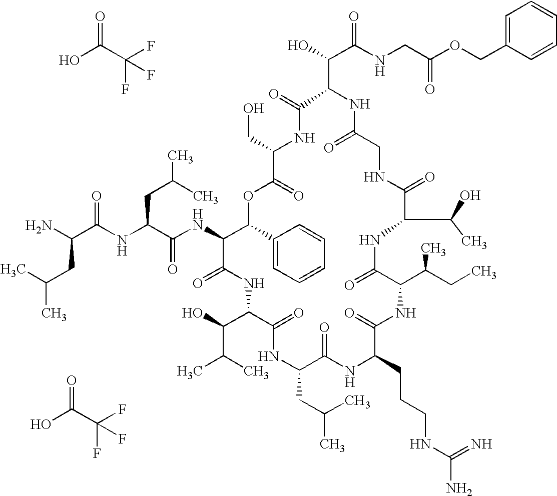

The Boc protecting group is removed from the compound of example 12A (19.0 mg, 11.59 μmol) according to procedure 1. Fine purification (Method 6) and freeze drying result in the title compound (16.0 mg, 84% of theory) as an amorphous solid.

HPLC (Method 15): Rₜ=17.81 min.

LC-MS (Method 10): Rₜ=1.64 min; MS (ESIpos.): m/z (%)=1425 (3) [M+H]⁺, 613 (100) [M+2H]²⁺; ESIneg: m/z (%)=1423 (18) [M−H]⁻, 711 (100) [M−2H]²⁻.

HR-TOF-MS (Method 11): C₆₇H₁₀₆N₁₅O₁₉ calc. 1424.7784, found 1424.7782 [M+H]⁺.

### Example 9

N^{4.10}-(2-{[(Benzyloxy)carbonyl]amino}ethyl)-D-leucyl-L-leucyl-[(3R)-3-hydroxy-L-phenylalanyl]-[(3R)-3-hydroxy-L-leucyl]-L-leucyl-D-arginyl-L-isoleucyl-L-allothreon-ylglycyl-[(3S)-3-hydroxy-L-asparaginyl]-L-serine C^{1.11}—O^{3.3}-lactone Bistrifluoroacetate

(N^{4.10}-(2-{[(Benzyloxy)carbonyl]amino}ethyl)lysobactin Bistrifluoroacetate)

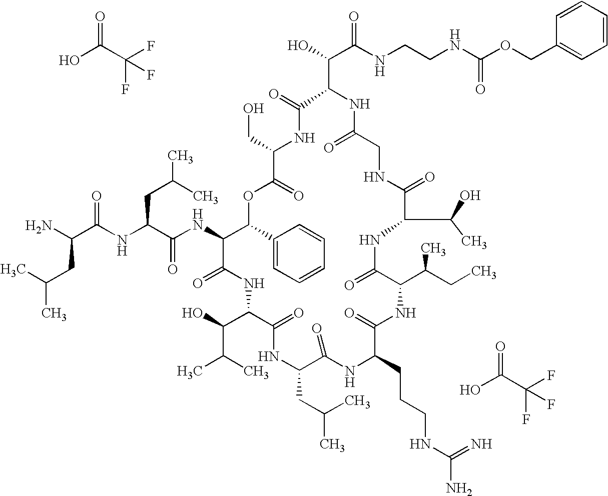

The Boc protecting group is removed from the compound of example 13A (10.0 mg, 6.00 μmol) according to procedure 1. Fine purification (Method 6) and freeze drying result in the title compound (10.0 mg, 99% of theory) as an amorphous solid.

HPLC (Method 15): Rₜ=18.07 min.

LC-MS (Method 10): Rₜ=1.64 min; MS (ESIpos.): m/z (%)=1455 (5) [M+H]⁺, 728 (100) [M+2H]²⁺; ESIneg: m/z (%)=1453 (15) [M−H]⁻, 726 (100) [M−2H]²⁻.

HR-TOF-MS (Method 11): C₆₈H₁₀₉N₁₆O₁₉ calc. 1453.8050, found 1453.8058 [M+H]⁺.

### Example 10

N^{4.10}-(2-Amino-2-oxoethyl)-D-leucyl-L-leucyl-[(3R)-3-hydroxy-L-phenylalanyl]-[(3R)-3-hydroxy-L-leucyl]-L-leucyl-D-arginyl-L-isoleucyl-L-allothreonylglycyl-[(3S)-3-hydroxy-L-asparaginyl]-L-serine C^{1.11}—O^{3.3}-lactone Bistrifluoroacetate

(N^{4.10}-(2-Amino-2-oxoethyl)lysobactin Bistrifluoroacetate)

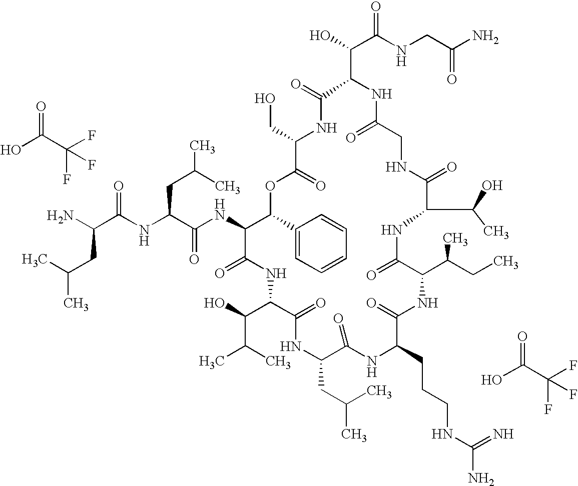

The Boc protecting group is removed from the compound of example 14A (11.0 mg, 7.11 μmol) according to procedure 1. Fine purification (Method 6) and freeze drying result in the title compound (7.2 mg, 65% of theory) as an amorphous solid.

HPLC (Method 15): Rₜ=15.60 min.

LC-MS (Method 10): Rₜ=1.48 min; MS (ESIpos.): m/z (%)=1334 (6) [M+H]⁺, 667 (100) [M+2H]²⁺; ESIneg: m/z (%)=1332 (20) [M−H]⁻, 665 (100) [M−2H]²⁻.

HR-TOF-MS (Method 11): C₆₀H₁₀N₁₆O₁₈ calc. 1333.7475, found 1333.7477 [M+H]⁺.

### Example 11

N^{4.10}-[2-(2-{[Bis(dimethylamino)methylene]amino}ethoxy)ethyl]-D-leucyl-L-leucyl-[(3R)-3-hydroxy-L-phenylalanyl]-[(3R)-3-hydroxy-L-leucyl]-L-leucyl-D-arginyl-L-isoleucyl-L-allothreonylglycyl-[(3S)-3-hydroxy-L-asparaginyl]-L-serine C^{1.11}—O^{3.3}-lactone Tristrifluoroacetate

(N^{4.10}-[2-(2-{[Bis(dimethylamino)methylene]amino}ethoxy)ethyl]lysobactin Tristrifluoroacetate)

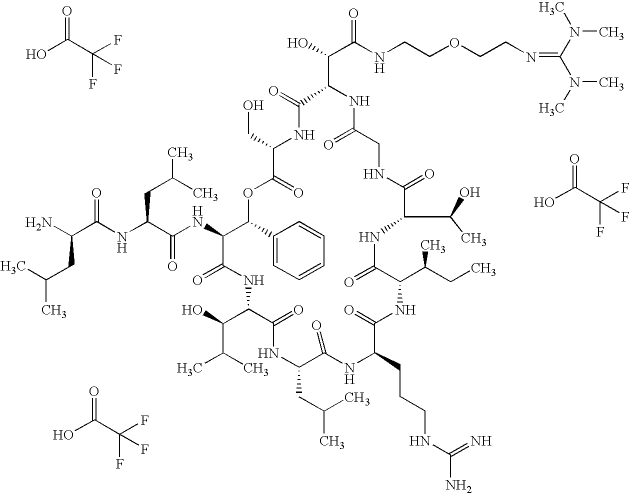

The Boc protecting group is removed from the compound of example 15A (4.0 mg, 2.22 μmol) according to procedure 1. Fine purification (Method 6) and freeze drying result in the title compound (2.7 mg, 67% of theory) as an amorphous solid.

HPLC (Method 15): Rₜ=16.00 min.

LC-MS (Method 10): Rₜ=1.40 min; MS (ESIpos.): m/z (%)=732 (48) [M+2H]²⁺, 488 (100); ESIneg: m/z (%)=1462 (20) [M−H]⁻, 1506 (100).

HR-TOF-MS (Method 11): C₆₇H₁₁₇N₁₈O₁₈ calc. 1461.8788, found 1461.8805 [M+H]⁺.

### Example 12

N^{4.10}-(2-Aminoethyl)-D-leucyl-L-leucyl-[(3R)-3-hydroxy-L-phenylalanyl]-[(3R)-3-hydroxy-L-leucyl]-L-leucyl-D-arginyl-L-isoleucyl-L-allothreonylglycyl-[(3S)-3-hydroxy-L-asparaginyl]-L-serine C^{1.11}—O^{3.3}-lactone Tristrifluoroacetate

(N^{4.10}-(2-Aminoethyl)lysobactin Tristrifluoroacetate)

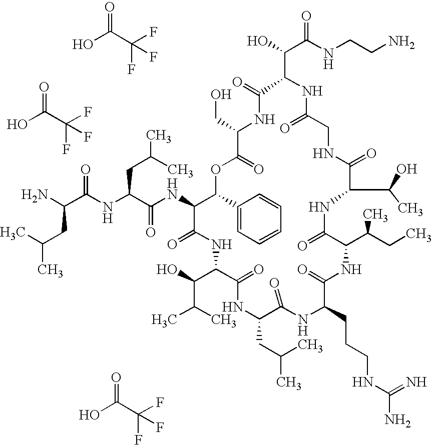

The benzyloxycarbonyl protecting group is removed from the compound of example 9 (8.0 mg, 4.76 μmol) by hydrogenolysis according to procedure 2. Fine purification (Method 6) and freeze drying result in the title compound (5.3 mg, 67% of theory) as an amorphous solid.

HPLC (Method 15): Rₜ=15.43 min.

LC-MS (Method 10): Rₜ=1.48 min; MS (ESIpos.): m/z (%)=1320 (3) [M+H]⁺, 660 (60) [M+2H]²⁺, 440 (100); ESIneg: m/z (%)=1318 (78) [M−H]⁻, 658 (100) [M−2H]²⁻.

HR-TOF-MS (Method 11): C₆₀H₁₀₃N₁₆O₁₇ calc. 1319.7682, found 1319.7725 [M+H]⁺.

### Example 13

N^{4.10}-(Carboxymethyl)-D-leucyl-L-leucyl-[(3R)-3-hydroxy-L-phenylalanyl]-[(3R)-3-hydroxy-L-leucyl]-L-leucyl-D-arginyl-L-isoleucyl-L-allothreonylglycyl-[(3S)-3-hydroxy-L-fi-asparaginyl]-L-serine C^{1.11}—O^{3.3}-lactone Bistrifluoroacetate

(N^{4.10}-(Carboxymethyl)lysobactin Bistrifluoroacetate)

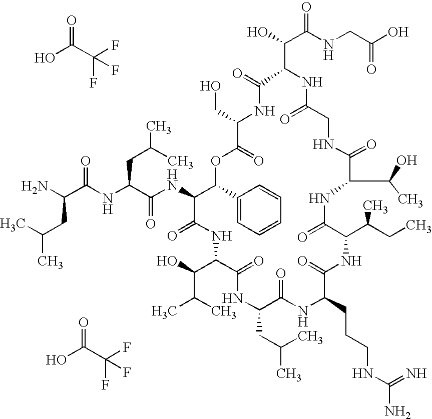

The benzyloxycarbonyl protecting group is removed from the compound of example 8 (4.0 mg, 2.42 μmol) by hydrogenolysis according to procedure 2. Fine purification (Method 6) and freeze drying result in the title compound (1.7 mg, 45% of theory) as an amorphous solid.

HPLC (Method 15): Rₜ=15.88 min.

LC-MS (Method 10): Rₜ=1.66 min; MS (ESIpos.): m/z (%)=1335 (4) [M+H]⁺, 668 (100) [M+2H]²⁺; ESIneg: m/z=1333 (32) [M−H]⁻, 666 (100) [M−2H]²⁻.

HR-TOF-MS (Method 11): C₆₀H₁₀₀N₁₅O₁₉ calc. 1334.7315, found 1334.7316 [M+H]⁺.

### B. Assessment of the Physiological Activity

The in vitro activity of the compounds of the invention can be shown in the following assays:

Determination of the Minimum Inhibitory Concentration (MIC):

The MIC is determined in the liquid dilution test in accordance with the NCCLS guidelines. Overnight cultures of Staphylococcus aureus 133, Enterococcus faecalis 27159, E. faecium 4147 and Streptococcus pneumoniae G9a are incubated with the described test substances in a 1:2 dilution series. The MIC determination is carried out with a cell count of 10⁵ microbes per ml in Isosensitest medium (Difco, Irvine/USA), with the exception of S. pneumoniae which is tested in BHI broth (Difco, Irvine/USA) with 10% bovine serum with a cell count of 10⁶ microbes per ml. The cultures are incubated at 37° C. for 18-24 hours, S. pneumoniae in the presence of 10% CO₂.

The MIC is defined as the lowest concentration of each substance at which no visible bacterial growth occurs any longer. The MIC values are reported in μg/ml.

Representative in vitro activity data of the compounds of the invention are shown in Table A:

TABLE A

| | | MIC | MIC |
|---|---|---|---|
| | MIC | S. pneumoniae | E. faecalis |
| | S. aureus 133 | G9a | ICB 27159 |
| Example No. | [μg/ml] | [μg/ml] | [μg/ml] |
| 2 | 1 | 2 | 16 |
| 3 | 0.25 | 0.5 | 2 |
| 8 | 0.5 | 2 | 16 |
| 9 | 0.5 | 2 | 16 |
| 10 | 0.25 | 1 | 2 |
| 12 | 0.5 | 2 | 8 |

The suitability of the compounds of the invention for the treatment of bacterial infections can be shown in the following animal model:

Systemic Infection with Staphylococcus aureus 133:

Cells of S. aureus 133 are grown overnight in BHI broth (Oxoid, N.Y./USA). The overnight culture is diluted 1:100 in fresh BHI broth and incubated for 3 hours. The cells which are then in the logarithmic phase of growth are centrifuged off and washed twice with buffered physiological saline. Then a cell suspension in saline is adjusted photometrically to an extinction of 50 units. After a dilution step (1:15), this suspension is mixed 1:1 with a 10% mucin solution. 0.25 ml of this infection solution are administered intraperitoneally per 20 g mouse (equivalent to 1×10⁶ microbes/mouse). Therapy takes place intraperitoneally or intravenously 30 minutes after the infection. Female CFW1 mice are used for the infection experiment. The survival of the animals is recorded over 6 days.

The properties of the compounds of the invention with regards to renal tolerability can be shown in the following animal model:

Mouse Model for the Determination of Nephrotoxic Effects:

Nephrotoxic side effects of the nonadepsipeptides are analyzed by histopathological examinations of the kidneys in mice and/or rats after multiple administration of a particular dosage. For this purpose, 5-6 animals are treated daily either intravenously (i.v.) or intraperitoneally (i.p.) with substances which are dissolved in aqueous solution or with addition of Solutol. Nephrotoxic effects are determined by optical microscopic assessment of hematoxylin and eosin (H&E) stained paraffin sections of the kidneys. A periodic acid Schiff (PAS) reaction is optionally carried out to visualize glycoproteins better. Nephrotoxic effects are specified semiquantitatively for each animal as severities of the tubular basophilia and degeneration/regeneration occurring (severities: 0=no effect; 1=minimal effect; 2=slight effect; 3=moderate effect; 4=severe lesions). The average severity of the tubular degeneration/regeneration as well as the incidence (number of affected animals) is calculated for each animal group or derivative. Renal changes going beyond this, such as tubular dilatation as well as necroses and the accumulation of necrotic material, are likewise listed.

Principle of the Determination of the Free Fraction Via Transil:

The method described here for determining the free fraction (fᵤ) of a test substance is divided into 2 parts:

a) Determination of the Transil®/buffer distribution ratio (MA_{buffer}) by incubating the test substance in a Transil®-buffer (pH 7.4) dispersion and subsequently determining the concentration in the dispersion and in the buffer supernatant.

b) Determination of the Transil®/plasma distribution ratio (MAₚₗₐₛₘₐ) by incubating the test substance in a Transil®-plasma dispersion and subsequently determining the concentration in the dispersion and in the plasma.

The quotient of the two distribution ratios yields fᵤ.

In the case of highly protein-bound substances, the plasma is usually diluted with isotonic phosphate buffer (pH 7.4) and then suspended with Transil®. The determination of fᵤ′ (free fraction in diluted plasma) in this diluted protein solution takes place in analogy to the determination of fᵤ. The free fraction in undiluted plasma is calculated from fᵤ′ and the dilution factor.

Concerning this method, compare also: Schuhmacher, Joachim; Kohlsdorfer, Christian; Buehner, Klaus; Brandenburger, Tim; Kruk, Renate, “High-throughput determination of the free fraction of drugs strongly bound to plasma proteins.” Journal of Pharmaceutical Sciences 2004, 93, 816-830.

Representative data from the determination of the free fraction for the compounds of the invention are shown in table B:

TABLE B

| | Free fraction | Free fraction |
|---|---|---|
| Example No. | (rat plasma) | (human plasma) |
| 2 | 13.7 | 9.9 |
| 3 | 12.9 | 5.1 |
| 4 | 6.8 | 2.4 |
| 6 | 7.0 | 2.6 |
| 1a (Lysobactin) | 0.76 | 0.81 |

Determination of the Membrane Affinity of a Test Substance after Distribution Between Transil® and Buffer (MA_{buffer}):

All incubations are carried out in suitable glass vessels, e.g. glass vials, ground-socket test tubes. In general the total volume is 0.5-5 ml, and the Transil® volume 10-100 μl. In cases where the membrane affinities are expected to be high, the Transil® dispersion can be diluted up to 20-fold with phosphate buffer of pH 7.4, e.g. Dulbecco's PBS. Phosphate buffer of pH 7.4 is provided in the incubation vessels, and the Transil® is pipetted in, after thorough mixing. The test substance is pipetted in at a concentration of, for example, 200 ng/mL, n=6. The proportion of organic solvent should be <2%. The mixtures are incubated at room temperature for 30 min, e.g. on a mini-shaker at an angle of about 45°, at about 400 rpm. In order to determine the 100% value at least one aliquot of, for example, 100 μl, is removed and the remaining mixture is centrifuged at about 1800 g for about 10 min. At least 2 aliquots (e.g. 100 μl) of the supernatant are removed from each sample for the determination of the concentration.

Determination of MAₚₗₐₛₘₐ in Undiluted or Diluted Plasma:

The total incubation volume and the added volume of Transil® depend on the expected free fraction. In general the total volume is 0.5-1 ml, and the Transil® volume is 10-100 μl. In cases where the free fractions are very low, the plasma of the species to be investigated is diluted, with isotonic buffer solution, pH 7.4, e.g. 10-400-fold, and the Transil® is then added. The subsequent procedure takes place as described above for the determination of the MA_{buffer} values.

Principle of the Determination of the Free Fraction Via Ultrafiltration:

The plasma of the species to be investigated is filtered through a semipermeable membrane. The substance concentration in the filtrate is measured and the free fraction fᵤ is calculated therefrom. The Centrifree micropartition system from Millipore/Amicon is used. The ultrafiltration membranes have a cut-off of 30 000 Da. 1 ml of plasma is doped with the substance in a concentration of about 1 μg/ml. The proportion of solvent should be <2%. After incubation at room temperature for 30 minutes, the plasma is pipetted into the ultrafiltration system and centrifuged at 1800 g for 10 minutes. The substance concentration in the ultrafiltrate (Cᵤ; unbound substance concentration) and in the plasma before centrifugation (C; total substance concentration) is measured. The free fraction is calculated by the formula: fᵤ (%)=Cᵤ/C*100.

The solubility of a compound is determined by methods known to a man of the art.

### C. Exemplary Embodiments of Pharmaceutical Compositions

The compounds of the invention can be converted into pharmaceutical preparations in the following ways:

Tablet:

Composition:

100 mg of the compound of Example 1, 50 mg of lactose (monohydrate), 50 mg of corn starch (native), 10 mg of polyvinylpyrrolidone (PVP 25) (BASF, Ludwigshafen, Germany) and 2 mg of magnesium stearate.

Tablet weight 212 mg. Diameter 8 mm, radius of curvature 12 mm.

Production:

A mixture of active ingredient, lactose and starch is granulated with a 5% solution (m/m) of the PVP in water. The granules are dried and then mixed with the magnesium stearate for 5 min. This mixture is compressed using a conventional tablet press (see above for format of the tablet). A compressive force of 15 kN is used as guideline for the compression.

Suspension which can be Administered Orally:

Composition:

1000 mg of the compound of Example 1, 1000 mg of ethanol (96%), 400 mg of Rhodigel (xanthan gum from FMC, Pennsylvania, USA) and 99 g of water.

10 ml of oral suspension are equivalent to a single dose of 100 mg of the compound of the invention.

Production:

The Rhodigel is suspended in ethanol, and the active ingredient is added to the suspension. The water is added while stirring. The mixture is stirred for about 6 h until the swelling of the Rhodigel is complete.

Solution which can be Administered Intravenously:

Composition:

100-200 mg of the compound of Example 1, 15 g of polyethylene glycol 400 and 250 g of water for injection.

Production:

The compound of Example 1 is dissolved together with polyethylene glycol 400 in the water with stirring. The solution is sterilized by filtration (pore diameter 0.22 μm) and dispensed under aseptic conditions into heat-sterilized infusion bottles. The latter are closed with infusion stoppers and crimped caps.

1. A compound of formula 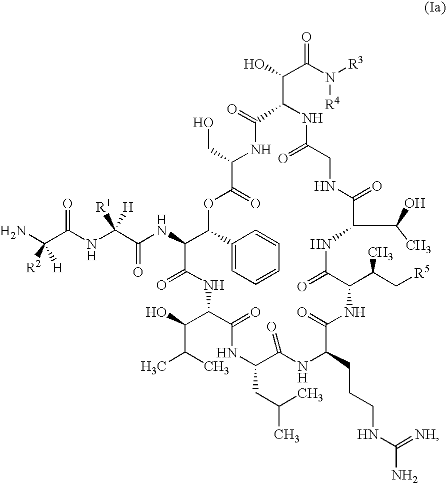
in which
R¹ represents 2-methylprop-1-yl, 2,2-dimethylprop-1-yl, 1-trimethylsilylmethyl or 3-pyridylmethyl,
whereby 3-pyridylmethyl may be substituted with a trifluoromethyl substituent,
R² represents 2-methylprop-1-yl, 2,2-dimethylprop-1-yl, 2,2-dimethylbut-1-yl, 2-ethyl-2-methylbut-1-yl, 2,2-diethylbut-1-yl, 2,2-dimethylpent-1-yl or 1-trimethylsilylmethyl,
R³ represents C₁-C₆-alkyl,
whereby alkyl may be substituted with 1 to 3 substituents, whereby the substituents are selected independently of one another from the group consisting of hydroxy, amino, hydroxycarbonyl, aminocarbonyl, {[bis-(dimethylamino)methylene]amino}ethoxy, phenyl, 5- or 6-membered heterocyclyl, 5- or 6-membered heteroaryl, benzyloxycarbonyl and benzyloxycarbonylamino,
R⁴ represents hydrogen, C₁-C₄-alkyl, cyclopropyl or cyclopropylmethyl,
R⁵ represents hydrogen or methyl,
or one of the salts thereof, the solvates thereof or the solvates of the salts thereof.

2. The compound of claim 1, corresponding to formula 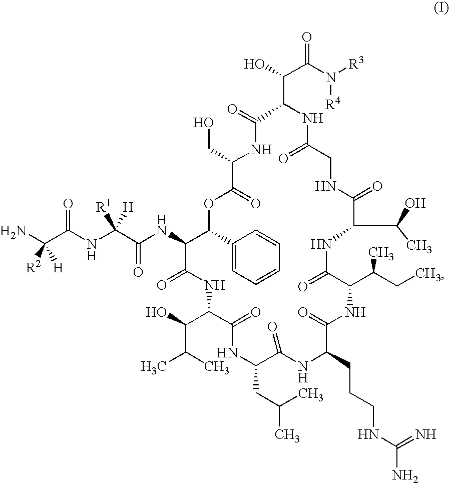
in which
R¹ represents 2-methylprop-1-yl, 2,2-dimethylprop-1-yl, 1-trimethylsilylmethyl or 3-pyridylmethyl,
whereby 3-pyridylmethyl may be substituted with a trifluoromethyl substituent,
R² represents 2-methylprop-1-yl, 2,2-dimethylprop-1-yl, 2,2-dimethylbut-1-yl, 2-ethyl-2-methylbut-1-yl, 2,2-diethylbut-1-yl, 2,2-dimethylpent-1-yl or 1-trimethylsilylmethyl,
R³ represents C₁-C₆-alkyl,
whereby alkyl may be substituted with 1 to 3 substituents, whereby the substituents are selected independently of one another from the group consisting of hydroxy, amino, hydroxycarbonyl, aminocarbonyl, {[bis-(dimethylamino)methylene]amino}ethoxy, phenyl, 5- or 6-membered heterocyclyl, 5- or 6-membered heteroaryl, benzyloxycarbonyl and benzyloxycarbonylamino,
R⁴ represents hydrogen, C₁-C₄-alkyl, cyclopropyl or cyclopropylmethyl,
or one of the salts thereof, the solvates thereof or the solvates of the salts thereof.

3. The compound of claim 1, whereby
R¹ represents 2-methylprop-1-yl, 2,2-dimethylprop-1-yl, 1-trimethylsilylmethyl or 3-pyridylmethyl,
whereby 3-pyridylmethyl may be substituted with a trifluoromethyl substituent,
R² represents 2-methylprop-1-yl, 2,2-dimethylprop-1-yl, 2,2-dimethylbut-1-yl, 2-ethyl-2-methylbut-1-yl, 2,2-diethylbut-1-yl, 2,2-dimethylpent-1-yl or 1-trimethylsilylmethyl,
R³ represents C₁-C₄-alkyl,
whereby alkyl may be substituted with 1 to 2 substituents, whereby the substituents are selected independently of one another from the group consisting of hydroxy, amino, hydroxycarbonyl, aminocarbonyl, {[bis-(dimethylamino)methylene]amino}ethoxy, phenyl, morpholin-4-yl, pyrid-2-yl, pyrid-3-yl, pyrid-4-yl, benzyloxycarbonyl and benzyloxycarbonylamino,
R⁴ represents hydrogen or methyl,
or one of the salts thereof, the solvates thereof or the solvates of the salts thereof.

4. The compound of claim 1, whereby
R¹ represents 2-methylprop-1-yl,
R² represents 2-methylprop-1-yl,
R³ represents C₁-C₃-alkyl,
whereby alkyl may be substituted with a substituent, whereby the substituent is selected from the group consisting of hydroxy, amino, hydroxycarbonyl, aminocarbonyl, {[bis(dimethylamino)methylene]amino}ethoxy, phenyl, morpholin-4-yl, pyrid-3-yl, benzyloxycarbonyl and benzyloxycarbonylamino,
R⁴ represents hydrogen or methyl,
or one of the salts thereof, the solvates thereof or the solvates of the salts thereof.

5. The compound of claim 1, whereby
R¹ represents 2,2-dimethylprop-1-yl,
R² represents 2,2-dimethylprop-1-yl,
R³ represents C₁-C₃-alkyl,
whereby alkyl may be substituted with a substituent, whereby the substituent is selected from the group consisting of hydroxy, amino, hydroxycarbonyl, aminocarbonyl, {[bis(dimethylamino)methylene]amino}ethoxy, phenyl, morpholin-4-yl, pyrid-3-yl, benzyloxycarbonyl and benzyloxycarbonylamino,
R⁴ represents hydrogen or methyl,
or one of the salts thereof, the solvates thereof or the solvates of the salts thereof.

6. A method for preparing a compound of formula (Ia) of claim 1, whereby a compound of formula 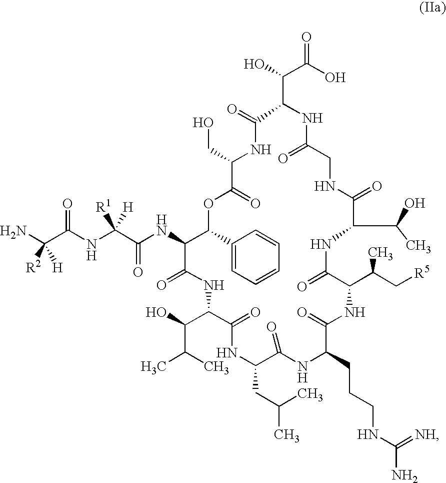
in which
R¹, R² and R⁵ have the meaning indicated in claim 1, is reacted with a compound of formula 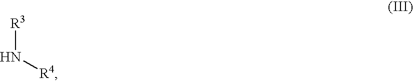
in which
R³ and R⁴ have the meaning indicated in claim 1.

7. The method of claim 6, whereby R⁵ represents methyl.

8. The method of claim 6, whereby
R¹ represents 2-methylprop-1-yl, 2,2-dimethylprop-1-yl, 1-trimethylsilylmethyl or 3-pyridylmethyl,
whereby 3-pyridylmethyl may be substituted with a trifluoromethyl substituent,
R² represents 2-methylprop-1-yl, 2,2-dimethylprop-1-yl, 2,2-dimethylbut-1-yl, 2-ethyl-2-methylbut-1-yl, 2,2-diethylbut-1-yl, 2,2-dimethylpent-1-yl or 1-trimethylsilylmethyl,
R³ represents C₁-C₄-alkyl,
whereby alkyl may be substituted with 1 to 2 substituents, whereby the substituents are selected independently of one another from the group consisting of hydroxy, amino, hydroxycarbonyl, aminocarbonyl, {[bis(dimethylamino)methylene]amino}ethoxy, phenyl, morpholin-4-yl, pyrid-2-yl, pyrid-3-yl, pyrid-4-yl, benzyloxycarbonyl and benzyloxycarbonylamino,
R⁴ represents hydrogen or methyl.

9. The method of claim 6, whereby
R¹ represents 2-methylprop-1-yl,
R² represents 2-methylprop-1-yl,
R³ represents C₁-C₃-alkyl,
whereby alkyl may be substituted with a substituent, whereby the substituent is selected from the group consisting of hydroxy, amino, hydroxycarbonyl, aminocarbonyl, {[bis(dimethylamino)methylene]amino}ethoxy, phenyl, morpholin-4-yl, pyrid-3-yl, benzyloxycarbonyl and benzyloxycarbonylamino,
R⁴ represents hydrogen or methyl.

10. The method of claim 6, whereby
R¹ represents 2,2-dimethylprop-1-yl,
R² represents 2,2-dimethylprop-1-yl,
R³ represents C₁-C₃-alkyl,
whereby alkyl may be substituted with a substituent, whereby the substituent is selected from the group consisting of hydroxy, amino, hydroxycarbonyl, aminocarbonyl, {[bis(dimethylamino)methylene]amino}ethoxy, phenyl, morpholin-4-yl, pyrid-3-yl, benzyloxycarbonyl and benzyloxycarbonylamino,
R⁴ represents hydrogen or methyl.

11. A compound of formula 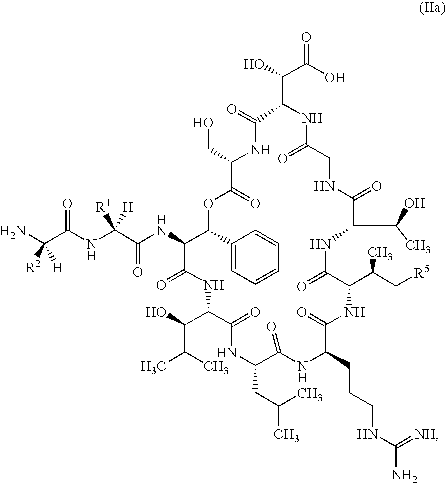
in which
R¹ represents 2-methylprop-1-yl, 2,2-dimethylprop-1-yl, 1-trimethylsilylmethyl or 3-pyridylmethyl,
whereby 3-pyridylmethyl may be substituted with a trifluoromethyl substituent,
R² represents 2-methylprop-1-yl, 2,2-dimethylprop-1-yl, 2,2-dimethylbut-1-yl, 2-ethyl-2-methylbut-1-yl, 2,2-diethylbut-1-yl, 2,2-dimethylpent-1-yl or 1-trimethylsilylmethyl,
R⁵ represents hydrogen or methyl,
or one of the salts thereof, the solvates thereof or the solvates of the salts thereof.

12. The compound of claim 11, corresponding to formula 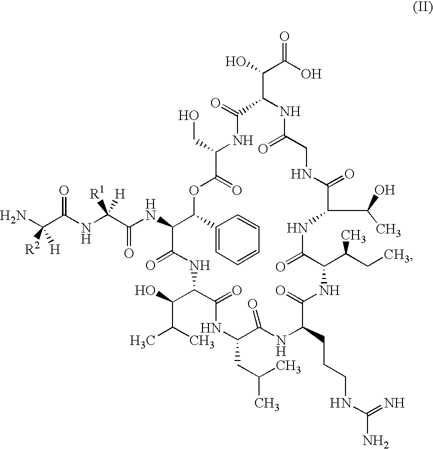
in which
R¹ represents 2-methylprop-1-yl, 2,2-dimethylprop-1-yl, 1-trimethylsilylmethyl or 3-pyridylmethyl,
whereby 3-pyridylmethyl may be substituted with a trifluoromethyl substituent,
R² represents 2-methylprop-1-yl, 2,2-dimethylprop-1-yl, 2,2-dimethylbut-1-yl, 2-ethyl-2-methylbut-1-yl, 2,2-diethylbut-1-yl, 2,2-dimethylpent-1-yl or 1-trimethylsilylmethyl,
or one of the salts thereof, the solvates thereof or the solvates of the salts thereof.

13. The compound of claim 11, whereby
R¹ represents 2-methylprop-1-yl,
R² represents 2-methylprop-1-yl,
or one of the salts thereof, the solvates thereof or the solvates of the salts thereof.

14. The compound of claim 11, whereby
R¹ represents 2,2-dimethylprop-1-yl,
R² represents 2,2-dimethylprop-1-yl,
or one of the salts thereof, the solvates thereof or the solvates of the salts thereof.

15. A method for preparing a compound of formula (IIa) of claim 11, whereby a compound of formula 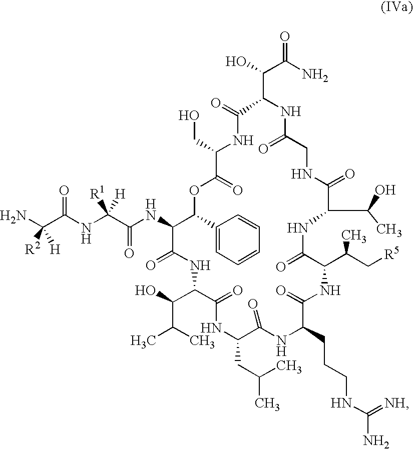
in which
R¹, R² and R⁵ have the meaning indicated in claim 11,
is hydrolyzed with an acid in a suitable solvent.

16. The method of claim 15, whereby said hydrolysis is carried out in a temperature range from room temperature to 50° C. under atmospheric pressure.

17. The method of claim 15, whereby said hydrolysis is carried out at room temperature and under atmospheric pressure.

18. The method of claim 15, whereby said solvent is selected from the group consisting of dioxane, tetrahydrofuran, water and mixtures thereof.

19. The method of claim 18, whereby said solvent is selected from the group consisting of a mixture of dioxane with water and a mixture of tetrahydrofuran with water.

20. The method of claim 15, whereby said acid is selected from the group consisting of the mineral acids and other strong acids.

21. The method of claim 20, whereby said acid is a mineral acid.

22. The method of claim 20, whereby said acid is selected from the group consisting of hydrochloric acid and methanesulfonic acid.

23. The method of claim 22, whereby said acid is hydrochloric acid.

24. The method of claim 15, whereby R⁵ represents methyl.

25. The method of claim 15, whereby R¹ and R² represent 2-methylprop-1-yl.

26. The method of claim 15 whereby R¹ and R² represent 2,2-dimethylprop-1-yl.

27. The compound of claim 1 for the treatment, prophylaxis or treatment and prophylaxis of diseases.

28. A method for manufacturing a medicament for the treatment, prophylaxis or treatment and prophylaxis of diseases using a compound of claim 1.

29. A method for manufacturing a medicament for the treatment, prophylaxis or treatment and prophylaxis of bacterial infections using a compound of claim 1.

30. A medicament comprising a compound of claim 1 in combination with an inert, nontoxic, pharmaceutically acceptable excipient.

31. The medicament of claim 30 for the treatment, prophylaxis or treatment and prophylaxis of bacterial infections.

32. A method for controlling bacterial infections in humans and animals by administering an antibacterially effective amount of at least one compound of claim 1, of a medicament of claim 30 or of a medicament obtained by the method of claim 28.
